(19) United States
(12) Patent Application Publication
(10) Pub. No.: US 2026/0286355 A1
(43) Pub. Date: Sep. 24, 2026

(54) DECOY RNA OLIGONUCLEOTIDES FOR INHIBITING YTHDF2

(71) Applicant: CATCHME RNA THERAPEUTICS LTD., Rehovot (IL)

(72) Inventors: Yael TSALENCHUCK, Ramat Gan (IL); Vladislav KRUPALNIK, Rehovot (IL); Shay GEULA, Rehovot (IL); Or YAKOV, Rehovot (IL)

(21) Appl. No.: 19/475,792

(22) PCT Filed: Apr. 16, 2024
(86) PCT No.: PCT/IL2024/050372

§ 371 Date: Oct. 16, 2025

Related U.S. Application Data
(60) Provisional application No. 63/459,652, filed on Apr. 16, 2023.

(51) Int. Cl. C12N 15/113; A61K 31/7115; A61K 31/712; A61K 31/7125; A61P 35/00

(52) U.S. Cl. CPC C12N 15/113; A61K 31/7115; A61K 31/712; A61K 31/7125; A61P 35/00; C12N 2310/13; C12N 2310/315; C12N 2310/321; C12N 2310/333; C12N 2310/346

(57) ABSTRACT

Nucleic acid molecule comprising a YTH domain-containing family protein 2 (YTHDF2) binding site are provided. Pharmaceutical compositions comprising the nucleic acid molecules and methods of inhibiting YTHDF2 or treating a disease with the nucleic acid molecules and compositions are also provided.

### REFERENCE TO AN ELECTRONIC SEQUENCE LISTING

The contents of the electronic sequence listing (CME-P-001-PCT.xml; Size: 139,469 bytes; and Date of Creation: Apr. 3, 2024) is herein incorporated by reference in its entirety.

### CROSS REFERENCE TO RELATED APPLICATIONS

This application claims the benefit of priority of U.S. Provisional Patent Application No. 63/459,652, filed Apr. 16, 2023, the contents of which are all incorporated herein by reference in their entirety.

### FIELD OF INVENTION

The present invention is in the field of epi-transcriptome modulation.

### BACKGROUND OF THE INVENTION

During the past decade, the emerging field of RNA posttranscriptional modifications (epitranscriptomics) has taken a big leap forward. The ability to manipulate different components of the epitranscriptome machinery holds great potential for new therapeutics. N6-methyladenosine (m6A) has been identified as the most abundant mRNA modification. It is present in eukaryotic species such as mammals, plants, insects, and yeast, as well as in certain viruses. The m6A modification is processed by a complex network of proteins that write, erase and bind to mRNA molecules carrying this modification. Recent research has shown that modulation of the m6A machinery can provide clinical benefits for different human pathologies like cancer and viral infections. YTHDF2 is one of the main m6A-RNA binding proteins that is involved in clearing m6A modified RNA. YTHDF2 was found to be overexpressed in various cytogenetic types of acute myeloid leukemia (AML). Depletion of YTHDF2 reduces the propagation and eradicates AML cells by inducing apoptosis. In addition, YTHDF2 was found to be upregulated in lung adenocarcinoma (LUAD). YTHDF2 knockdown significantly inhibited LUAD tumorigenesis and promoted survival. Mechanistically, YTHDF2 binds directly to the m6A-modified site on the AXIN1 mRNA, leading to its destabilization. YTHDF2 promoted AXIN1 mRNA decay and subsequently activated Wnt/β-catenin signaling. Moreover, YTHDF2 depletion was demonstrated to trigger apoptosis in triple-negative breast cancer cells and tumors. New method of inhibiting YTHDF2 binding to m6A and molecules for performing these methods are therefore greatly needed.

### SUMMARY OF THE INVENTION

The present invention provides nucleic acid molecule comprising a YTH domain-containing family protein 2 (YTHDF2) binding site. Pharmaceutical compositions comprising the nucleic acid molecules and methods of inhibiting YTHDF2 or treating a disease with the nucleic acid molecules and compositions are also provided.

According to a first aspect, there is provided a nucleic acid molecule comprising a YTH domain-containing family protein 2 (YTHDF2) binding site and a 5′ enhancer motif located 5′ to the binding site, wherein the 5′ enhancer motif is selected from GGGAAACU, CUGCUGCU, GGAGAGCU, UGGCCACC and SEQ ID NO: 44 and wherein the molecule comprises 13-100 nucleotides.

According to another aspect, there is provided a nucleic acid molecule comprising a YTHDF2 binding site, wherein the molecule comprises 10-100 nucleotides and a phosphorothioate (PS) modified backbone.

According to some embodiments, the nucleic acid molecule comprises a 5′ enhancer motif located 5′ to the binding site, wherein the 5′ enhancer motif is selected from GGGAAACU, CUGCUGCU, GGAGAGCU, UGGCCACC and SEQ ID NO: 44.

According to some embodiments, the 5′ enhancer motif is GGGAAACU.

According to some embodiments, the binding site consists of a sequence selected from GGACU, GAACU, GAACC, GAACA, AGACU, AGACC, AGACA, GGACC, GGACA, AAACU, AAACA, AAACG, UAACU, UAACA, UAACG, UGACU, UGACA and UGACG and wherein the central adenosine in the binding site is a N6 methyladenosine.

According to some embodiments, the binding site consists of GGACU and the adenosine is an N6 methyladenosine.

According to some embodiments, the 5′ enhancer motif and the binding site are separated by a first spacer.

According to some embodiments, the first spacer comprises 1-6 nucleotides.

According to some embodiments, the molecule is a single stranded RNA molecule.

According to some embodiments, the molecule is a single stranded DNA molecule.

According to some embodiments, the molecule is a chimeric molecule comprising both DNA and RNA bases.

According to some embodiments, the binding site is DNA bases.

According to some embodiments, all non-binding site bases are RNA bases.

According to some embodiments, the nucleic acid molecule comprises a chemically modified backbone.

According to some embodiments, the chemically modified backbone comprises a PS linkage.

According to some embodiments, the nucleic acid molecule comprises a fully PS-modified backbone.

According to some embodiments, the nucleic acid molecule comprises a PS-modified backbone in which the binding site is not PS modified.

According to some embodiments, the nucleic acid molecule further comprises at least one 2′O methyl modified base.

According to some embodiments, all RNA bases are 2′O methyl modified.

According to some embodiments, the nucleic acid molecule comprises the nucleotide sequence CUGGGAAACUGGmACUGAG (SEQ ID NO: 66), wherein mA is a N6 methyladenosine.

According to some embodiments, the nucleic acid molecule comprises a sequencing consisting of SEQ ID NO: 66.

According to some embodiments, the nucleic acid molecule consists of SEQ ID NO: 67.

According to some embodiments, the nucleic acid molecule further comprises a 3′ enhancer motif, wherein the 3′ enhancer motif is selected from SEQ ID NO: 45-49.

According to some embodiments, the 3′ enhancer motif and the binding site are separated by a second spacer.

According to some embodiments, the second spacer comprises 1-6 nucleotides.

According to some embodiments, the nucleic acid molecule further comprises a 5′ terminal spacer, a 3′ terminal spacer or both.

According to some embodiments, the nucleic acid molecule comprises 2, 3 or 4 binding sites.

According to some embodiments, the binding sites comprise the same or different sequences.

According to some embodiments, the 2, 3 or 4 binding sites are separated by third spacers.

According to some embodiments, the third spacer comprises 1-6 nucleotides.

According to some embodiments, the nucleic acid molecule comprises a sequence selected from SEQ ID NO: 21-25, 28-30, 32-42 and 65-66.

According to some embodiments, the sequence of the nucleic acid molecule consists of a sequence selected from SEQ ID NO: 21-25, 28-30, 32-42 and 65-66.

According to some embodiments, the nucleic acid molecule, comprises a sequence selected from SEQ ID NO: 21-25, 33-42 and 66.

According to some embodiments, the sequence of the nucleic acid molecule consists of a sequence selected from SEQ ID NO: 21-25, 33-42 and 66.

According to some embodiments, the nucleic acid molecule comprises a sequence selected from SEQ ID NO: 1-9, 11-42, 51-55 and 65-66.

According to some embodiments, the sequence of the nucleic acid molecule consists of a sequence selected from SEQ ID NO: 1-9, 11-42, 51-55 and 65-66.

According to some embodiments, the nucleic acid molecule comprises a sequence selected from SEQ ID NO: 1-3, 5-6, 8-9, 11-26, 28-42 51-55 and 66.

According to some embodiments, the sequence of the nucleic acid molecule consists of a sequence selected from SEQ ID NO: 1-3, 5-6, 8-9, 11-26, 28-42 51-55 and 66.

According to another aspect, there is provided a pharmaceutical composition comprising a nucleic acid molecule of the invention and a pharmaceutically acceptable carrier excipient or adjuvant.

According to some embodiments, the pharmaceutical composition is formulated for administration to a subject.

According to some embodiments, the pharmaceutical composition is for use in inhibiting YTHDF2.

According to some embodiments, the inhibiting is inhibiting YTHDF2 mediated degradation of a m6A containing mRNA.

According to some embodiments, the pharmaceutical composition is for use in treating a subject suffering from a disease or condition characterized by YTHDF2 mediated mRNA instability or degradation.

According to some embodiments, the disease or condition is selected from cancer, or viral infection.

According to some embodiments, the cancer is selected from lung cancer, liver cancer, leukemia, bladder cancer, pancreatic cancer, prostate cancer and breast cancer.

According to some embodiments, the cancer is selected from lung cancer, liver cancer brain cancer, renal cancer, muscle cancer, uterine cancer, cervical cancer, head and neck cancer, pancreatic cancer and breast cancer.

According to some embodiments, the cancer is selected from lung cancer, liver cancer and breast cancer.

According to some embodiments, the viral infection is selected from a Kaposi's Sarcoma-associated Herpesvirus (KSHV), Human Cytomegalovirus (HCMV), Human Immunodeficiency Virus 1 (HIV-1) and influenza.

According to another aspect, there is provided a method of inhibiting YTHDF2, the method comprising contacting the YTHDF2 with a nucleic acid molecule of the invention or a pharmaceutical composition of the invention, thereby inhibiting YTHDF2.

According to some embodiments, the inhibiting is inhibiting YTHDF2 mediated degradation of a m6A containing mRNA.

According to another aspect, there is provided a method of treating a disease or condition characterized by YTHDF2 mediated mRNA instability or degradation in a subject in need thereof, the method comprising administering to the subject a pharmaceutical composition of the invention, thereby treating a disease or condition.

According to some embodiments, YTHDF2 mediated mRNA instability or degradation causes, sustains or enhances the disease or condition.

According to some embodiments, the disease or condition is selected from cancer, or viral infection.

According to some embodiments, the cancer is selected from lung cancer, liver cancer, leukemia, bladder cancer, pancreatic cancer, prostate cancer and breast cancer.

According to some embodiments, the cancer is selected from lung cancer, liver cancer brain cancer, renal cancer, muscle cancer, uterine cancer, cervical cancer, head and neck cancer, pancreatic cancer and breast cancer.

According to some embodiments, the cancer is selected from lung cancer, liver cancer and breast cancer.

According to some embodiments, the viral infection is selected from a Kaposi's Sarcoma-associated Herpesvirus (KSHV), Human Cytomegalovirus (HCMV), Human Immunodeficiency Virus 1 (HIV-1) and influenza.

According to some embodiments, the method further comprises measuring YTHDF2 levels or mRNA instability, or degradation is a sample containing disease cells from the subject, wherein the presence of YTHDF2 levels or mRNA instability or degradation above a predetermined threshold indicates the subject is suitable to be treated by a method of the invention.

According to another aspect, there is provided a method of determining suitability of a subject in need thereof to be treated by a method of the invention, the method comprising, receiving a sample containing disease cells from the subject, and measuring YTHDF2 levels or mRNA instability or degradation in the sample, wherein the presence of YTHDF2 levels or mRNA instability or degradation above a predetermined threshold indicates the subject is suitable to be treated by a method of the invention, thereby determining suitability.

According to some embodiments, the method further comprises administering a pharmaceutical composition of the invention to a subject determined to be suitable.

According to some embodiments, the measured mRNA instability or degradation is YTHDF2 mediated.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1: A line graph of results of the modified ELISA showing relative absorbance at increasing concentrations of the control RNA oligo (SEQ ID NO: 1) and RNA oligos containing alternative binding sites (SEQ ID NO: 2-6).

FIG. 2: A line graph of results of the modified ELISA showing relative absorbance at increasing concentrations of the control RNA oligo (SEQ ID NO: 1) and an RNA oligo of the invention (SEQ ID NO: 22).

FIGS. 3A-3B: Bar graphs of percent viability of (3A) MDA-MB-468 cells and (3B) MDA-MB-231 cells after administration of the oligos (200 nM) listed in Table 8. A scrambled (SCRM) irrelevant RNA was used as a negative control. **** is a p-val<0.001.

FIGS. 4A-4B: Line graphs of percent viability of MDA-MB-231 cells in response to increasing doses of (4A) scrambled oligos containing only PS linkages (SCRM-PS), PS linkages and alternating 2′F modification and 2′Ome modifications (SCRM-f/o), or PS linkages and 5 nucleotide blocks of 2′F modification and 2′Ome modifications (SCRM-bf/o) or (4B) scrambled oligos containing only PS linkages (SCRM-PS), PS linkages and 2′Ome modification (SCRM-O), or PS linkages and 2-methoxyethyl modification (SCRM-MOE).

FIG. 5: Line graph of percent viability of MDA-MB-231 cells in response to increasing doses of various oligos with PS linkages and 2′Ome or 2-methoxyethyl modifications.

FIGS. 6A-6B: Line graphs of percent viability in (6A) MDA-MB-231 cells and (6B) patient derived cell line MAXFTN in response to increasing doses of CMT-124.

FIG. 7: Line graphs of percent survival in healthy breast cell lines MCF10 and MCF12 in response to increasing doses of CMT-124.

FIGS. 8A-8B: Bar graphs of secretion of (8A) IL-6 and (8B) TNF-alpha from human PBMCs in response to various treatments.

FIGS. 9A-9D: Line graphs of Ln (percent remaining) of (9A, 9C) Patisiran or (9B, 9D) CMT-124 cultured over time in (9A-9B) plasma or (9C-9D) S9 liver extract.

### DETAILED DESCRIPTION OF THE INVENTION

The present invention, in some embodiments, provides nucleic acid molecule comprising a YTH domain-containing family protein 2 (YTHDF2) binding site and a 5′ enhancer motif. Nucleic acid molecules comprising a YTHDF2 binding site and a phosphorothioate (PS) modified backbone are also provided. Pharmaceutical compositions comprising the nucleic acid molecules and methods of inhibiting YTHDF2 or treating a disease or condition with the nucleic acid molecules and compositions are also provided. Methods of determining suitability to be treated by a method of the invention are also provided.

The invention is based, at least in part, on the surprising finding that placement of several specific motifs 5′ to the YTHDF2 canonical binding site greatly enhances YTHDF2 binding. This allows for the construction of highly effective decoy molecules that bind YTHDF2 with very high affinity. Similarly, it was found that decoy molecules with a phosphorothioate (PS) modified backbone also exhibited greatly enhanced YTHDF2 binding. Several other modifications, such as addition of a 3′ motif, repetitions of the binding site and the use of other modifications were also found to increase YTHDF2 binding but to a lesser extent. These decoy molecules are sufficiently efficient binders of YTHDF2 such as to sequester this epi-transcriptome reader and render mRNA targets of YTHDF2 more stable and less prone to degradation.

By a first aspect, there is provided a nucleic acid molecule comprising a YTH domain-containing family protein 2 (YTHDF2) binding site and an enhancer motif.

By another aspect, there is provided a nucleic acid molecule comprising a YTHDF2 binding site and a phosphorothioate (PS) modified backbone.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C).

The term “nucleic acid molecule” includes but is not limited to single-stranded RNA (ssRNA), double-stranded RNA (dsRNA), single-stranded DNA (ssDNA), double-stranded DNA (dsDNA), small RNA such as miRNA, siRNA and other short interfering nucleic acids, snoRNAs, snRNAs, tRNA, piRNA, tnRNA, small rRNA, hnRNA, lncRNA, circulating nucleic acids, fragments of genomic DNA or RNA, degraded nucleic acids, ribozymes, viral RNA or DNA, nucleic acids of infectious origin, amplification products, modified nucleic acids, plasmidical or organellar nucleic acids and artificial nucleic acids such as oligonucleotides. In some embodiments, the nucleic acid molecule is a DNA molecule. In some embodiments, the nucleic acid molecule is a single strand DNA molecule. In some embodiments, the nucleic acid molecule is RNA. In some embodiments, the RNA molecule is a single stranded RNA molecule. In some embodiments, the RNA molecule comprises a 5′ cap. In some embodiments, the RNA molecule does not comprise a 5′ cap. In some embodiments, the RNA comprises 3′ poly-adenylation. In some embodiments, the RNA does not comprise 3′ poly-adenylation. In some embodiments, the nucleic acid molecule is a polynucleotide. In some embodiments, the nucleic acid molecule is an oligonucleotide. In some embodiments, the RNA is not an mRNA. In some embodiments, the RNA does not comprise an open reading frame. In some embodiments, the nucleic acid molecule is a decoy molecule. In some embodiments, a decoy is a YTHDF2 binding molecule. In some embodiments, the nucleic acid molecule is an artificial molecule. In some embodiments, the nucleic acid molecule is a synthetic molecule. In some embodiments, the nucleic acid molecule is not found in nature.

In some embodiments, the nucleic acid molecule comprises at least 5, 7, 9, 10, 11, 12, 13, 14, 15, 16, 17, 18, 19, 20, 21, 22, 23, 24 or 25 nucleotides. Each possibility represents a separate embodiment of the invention. In some embodiments, the nucleic acid molecule comprises at least 10 nucleotides. In some embodiments, the nucleic acid molecule comprises at least 13 nucleotides. In some embodiments, the nucleic acid molecule comprises at least 15 nucleotides. In some embodiments, the nucleic acid molecule comprises at least 16 nucleotides. In some embodiments, the nucleic acid molecule comprises at least 18 nucleotides. In some embodiments, the nucleic acid molecule comprises at most 18, 20, 25, 26, 27, 28, 29, 30, 31, 32, 33, 34, 35, 40, 45, 50, 55, 60, 65, 70, 75, 80, 85, 90, 95, 100, 125, 150, 175, 200, 225, 250, 275, 300, 350, 400, 450, 500, 550, 600, 650, 700, 750, 800, 850, 900, 950 or 1000 nucleotides. Each possibility represents a separate embodiment of the invention. In some embodiments, the nucleic acid molecule comprises at most 18 nucleotides. In some embodiments, the nucleic acid molecule comprises at most 33 nucleotides. In some embodiments, the nucleic acid molecule comprises at most 35 nucleotides. In some embodiments, the nucleic acid molecule comprises at most 45 nucleotides. In some embodiments, the nucleic acid molecule comprises at most 50 nucleotides. In some embodiments, the nucleic acid molecule comprises at most 100 nucleotides. In some embodiments, the nucleic acid molecule comprises between 8-18, 8-20, 8-30, 8-35, 8-40, 8-45, 8-50, 8-75, 8-100, 8-150, 8-200, 10-18, 10-20, 10-30, 0-35, 10-40, 10-45, 10-50, 10-75, 10-100, 10-150, 10-200, 13-18, 13-20, 13-30, 13-35, 13-40, 13-45, 13-50, 13-75, 13-100, 13-150, 13-200, 15-18, 15-20, 15-30, 15-35, 15-40, 15-45, 15-50, 15-75, 15-100, 15-150, 15-200, 16-18, 16-20, 16-30, 16-35, 16-40, 16-45, 16-50, 16-75, 16-100, 16-150, 16-200, 18-20, 18-30, 18-35, 18-40, 18-45, 18-50, 18-75, 18-100, 18-150, or 18-200 nucleotides. Each possibility represents a separate embodiment of the invention. In some embodiments, the nucleic acid molecule comprises between 10-100 nucleotides. In some embodiments, the nucleic acid molecule comprises between 13-100 nucleotides.

YTH domain-containing family protein 2 (YTHDF2) is also known as YTH N6-methyladenosine RNA binding protein 2 and is a member of the YTH domain containing superfamily. It is known to bind mRNA at N-6 methyladenosine (m6A) residues. In some embodiments, the amino acid sequence of human YTHDF2 comprises or consists of SEQ ID NO: 63. In some embodiments, the YTHDF2 binding site comprises a m6A residue. In some embodiments, the binding site comprises or consists of a sequence selected from GGACU, GAACU, GAACC, GAACA, AGACU, AGACC, AGACA, GGACC and GGACA. In some embodiments, the binding site comprises or consists of a sequence selected from GGACU, GAACU, GAACC, GAACA, AGACU, AGACC, AGACA, GGACC, GGACA, AAACU, AAACA, AAACG, UAACU, UAACA, UAACG, UGACU, UGACA and UGACG. In some embodiments, the central adenosine in the binding site is an N6 methyladenosine. In some embodiments, N6 methyladenosine is DNA methyladenosine. In some embodiments, N6 methyladenosine is RNA methyladenosine. In some embodiments, the binding site comprises or consists of a sequence selected from GGm6ACU, GAm6ACU, GAm6ACC, GAm6ACA, AGm6ACU, AGm6ACC, AGm6ACA, GGm6ACC, GGm6ACA, AAm6ACU, AAm6ACA, AAm6ACG, UAm6ACU, UAm6ACA, UAm6ACG, UGm6ACU, UGm6ACA and UGm6ACG. In some embodiments, the binding site comprises or consists of GGACU where the central adenosine is a N6 methyladenosine. In some embodiments, the binding site comprises or consists of GGm6ACU. In some embodiments, GGm6ACU is the canonical YTHDF2 binding site.

In some embodiments, the nucleic acid molecule comprises a chemically modified backbone. In some embodiments, the nucleic acid molecule is a chimeric molecule. In some embodiments, the modification increases stability of the RNA. In some embodiments, the modification increases half-life of the RNA. In some embodiments, the modification decreases degradation of the RNA. In some embodiments, the modification decreases cleavage of the RNA. In some embodiments, the modification decreases immunogenicity of the RNA. In some embodiments, the modification decreases off-target effects. Chemical modifications of RNA are well known in the art and include, for example, 2-O-methyl analogs, 2′-fluoro analogs, 2′-MOE, 2′-OME, phosphorothioate (PS) and phosphorolthiolate, and any such modification may be employed. In some embodiments, the modification is a 2′O methyl(2′-OME). In some embodiments, a 2′O methyl modification is 2′O methylation. In some embodiments, the modification is PS. In some embodiments, the modification is phosphorolthiolate. In some embodiments, the modification is a combination of modifications. In some embodiments, the molecule comprises PS and 2′O methyl modification. In some embodiments, the backbone comprises at least one PS linkage. In some embodiments, the backbone comprises a plurality of PS linkages. In some embodiments, a plurality is at least 2, 3, 4, 5, 6, 7, 8, 9, 10, 11, 12, 13, 14, 15, 16, 17, 18, 19, or 20 PS linkages. Each possibility represents a separate embodiment of the invention. In some embodiments, all nucleotide linkages are PS linkages. In some embodiments, the nucleic acid comprises a fully PS modified backbone. In some embodiments, the molecule comprises a PS-modified backbone in which the binding stie is not PS-modified. In some embodiments, the molecule comprises a PS-modified backbone in which the m6A nucleotide is not modified by a PS linkage. In some embodiments, the entire molecule is PS-modified except for the binding site. In some embodiments, the entire molecule is PS-modified except for the m6A nucleotide. In some embodiments, the entire molecule is chemically modified except for the binding site. In some embodiments, the entire molecule is chemically modified except for the m6A nucleotide. In some embodiments, at least 2, 3, 4, 5, 6, 7, 8, 9, 10, 11, 12, 13, 14, 15, 16, 17, 18, 19, or 20 bases are 2′O methyl modified. Each possibility represents a separate embodiment of the invention. In some embodiments, all bases are 2′O methylated. In some embodiments, all bases except for the binding site are 2′O methylated. In some embodiments, all bases except for the m6A nucleotide are 2′O methylated. In some embodiments, all RNA bases are 2′O methylated.

In some embodiments, the nucleic acid molecule is a chimeric molecule. In some embodiments, the nucleic acid molecule comprises DNA bases and RNA bases. In some embodiments, the entire molecule is a DNA molecule. In some embodiments, the entire molecule is an RNA molecule. In some embodiments, the entire molecule is a DNA molecule but the binding site comprises RNA bases. In some embodiments, the entire molecule is a DNA molecule but the binding site is completely RNA bases. In some embodiments, the entire molecule is a DNA molecule but the m6A is an RNA base. In some embodiments, the entire molecule is an RNA molecule but the binding site comprises DNA bases. In some embodiments, the entire molecule is a RNA molecule but the binding site is completely DNA bases. In some embodiments, the binding site is DNA bases. In some embodiments, the entire binding site is DNA bases. In some embodiments, all non-binding site bases are RNA bases.

In some embodiments, the nucleic acid molecule comprises an enhancer motif. In some embodiments, the enhancer motif is a 5′ enhancer motif. In some embodiments, the enhancer motif is located 5′ to the binding site. In some embodiments, the enhancer motif is a plurality of enhancer motifs. In some embodiments, a plurality is at least 2, 3, 4, 5, 6, 7, 8, 9 or 10 enhancer motifs. Each possibility represents a separate embodiment of the invention. In some embodiments, the enhancer motif is a plurality of 5′ enhancer motifs. In some embodiments, a plurality is at least 2, 3, 4, 5, 6, 7, 8, 9 or 10 5′ enhancer motifs. In some embodiments, the nucleic acid molecule comprises at least one 5′ enhancer motif.

In some embodiments, the enhancer motif comprises GGGAAACU. In some embodiments, the enhancer motif consists of GGGAAACU. In some embodiments, the 5′ enhancer motif comprise or consists of GGGAAACU. In some embodiments, the enhancer motif comprises UCAGAGUC. In some embodiments, the enhancer motif consists of UCAGAGUC. In some embodiments, the 5′ enhancer motif comprise or consists of UCAGAGUC. In some embodiments, the enhancer motif comprises CUGCUGCU. In some embodiments, the enhancer motif consists of CUGCUGCU. In some embodiments, the 5′ enhancer motif comprise or consists of CUGCUGCU. In some embodiments, the enhancer motif comprises GGAGAGCU. In some embodiments, the enhancer motif consists of GGAGAGCU. In some embodiments, the 5′ enhancer motif comprise or consists of GGAGAGCU. In some embodiments, the enhancer motif comprises UGGCCACC. In some embodiments, the enhancer motif consists of UGGCCACC. In some embodiments, the 5′ enhancer motif comprise or consists of UGGCCACC. In some embodiments, the enhancer motif comprises AUGAGUCCAAC (SEQ ID NO: 43). In some embodiments, the enhancer motif consists of SEQ ID NO: 43. In some embodiments, the 5′ enhancer motif comprise or consists of SEQ ID NO: 43. In some embodiments, the enhancer motif comprises AUGAGUCCAAC (SEQ ID NO: 44). In some embodiments, the enhancer motif consists of SEQ ID NO: 44. In some embodiments, the 5′ enhancer motif comprise or consists of SEQ ID NO: 44. In some embodiments, the enhancer motif is selected from GGGAAACU, CUGCUGCU, GGAGAGCU, UGGCCACC and SEQ ID NO: 44. In some embodiments, the 5′ enhancer motif is selected from GGGAAACU, CUGCUGCU, GGAGAGCU, UGGCCACC and SEQ ID NO: 44.

In some embodiments, each binding site comprises a 5′ enhancer motif. In some embodiments, the molecule comprises 2, 3, 4, 5, 6, 7, 8, 9 or 10 YTHDF2 binding sites. Each possibility represents a separate embodiment of the invention. In some embodiments, the molecule comprises a plurality of YTHDF2 binding sties. In some embodiments, the molecule comprises 2 YTHDF2 binding sites. In some embodiments, the molecule comprises 3 YTHDF2 binding sites. In some embodiments, the molecule comprises 4 YTHDF2 binding sites. In some embodiments, the molecule comprises 1~4 binding sites. In some embodiments, the molecule comprises 2-4 binding sites. In some embodiments, at least one of the YTHDF2 binding sites comprises an enhancer motif. In some embodiments, at least one of the YTHDF2 binding sites comprises a 5′ enhancer motif. In some embodiments, each YTHDF2 binding site comprises an enhancer motif. In some embodiments, each YTHDF2 binding site comprises a 5′ enhancer motif. In some embodiments, the binding sites are all the same within a molecule. In some embodiments, the binding sites comprise the same sequence. In some embodiments, at least two of the binding sites in the molecule comprise different sequences. In some embodiments, all the binding sites comprise different sequences. In some embodiments, the binding sites comprise different sequences.

In some embodiments, the enhancer motif is a 3′ enhancer motif. In some embodiments, the enhancer motif is located 3′ to the binding site. In some embodiments, the enhancer motif is a plurality of 3′ enhancer motifs. In some embodiments, a plurality is at least 2, 3, 4, 5, 6, 7, 8, 9 or 10 3′ enhancer motifs. Each possibility represents a separate embodiment of the invention. In some embodiments, the nucleic acid molecule comprises at least one 3′ enhancer motif.

In some embodiments, the enhancer motif comprises AGACCUGGCU (SEQ ID NO: 45). In some embodiments, the enhancer motif consists of SEQ ID NO: 45. In some embodiments, the 3′ enhancer motif comprise or consists of SEQ ID NO: 45. In some embodiments, the enhancer motif comprises GAGCAUCUGC (SEQ ID NO: 46). In some embodiments, the enhancer motif consists of SEQ ID NO: 46. In some embodiments, the 3′ enhancer motif comprise or consists of SEQ ID NO: 46. In some embodiments, the enhancer motif comprises GCUGCUGCUGCU (SEQ ID NO: 47). In some embodiments, the enhancer motif consists of SEQ ID NO: 47. In some embodiments, the 3′ enhancer motif comprise or consists of SEQ ID NO: 47. In some embodiments, the enhancer motif comprises UGUGCAGCCCUC (SEQ ID NO: 48). In some embodiments, the enhancer motif consists of SEQ ID NO: 48. In some embodiments, the 3′ enhancer motif comprise or consists of SEQ ID NO: 48. In some embodiments, the enhancer motif comprises CUGGGCCUGGGC (SEQ ID NO: 49). In some embodiments, the enhancer motif consists of SEQ ID NO: 49. In some embodiments, the 3′ enhancer motif comprise or consists of SEQ ID NO: 49. In some embodiments, the enhancer motif is selected from SEQ ID NO: 45-49. In some embodiments, the 3′ enhancer motif is selected from SEQ ID NO: 45-49.

In some embodiments, the enhancer motif and the binding site are separated by a spacer. In some embodiments, the 5′ enhancer motif is separated from the binding site by a first spacer. In some embodiments, the 3′ enhancer motif is separated from the binding site by a second spacer. In some embodiments, a spacer comprises at least 1, 2, 3, 4, 5, or 6 nucleotides. Each possibility represents a separate embodiment of the invention. In some embodiments, a spacer comprises at least 1 nucleotide. In some embodiments, a spacer comprises at least 2 nucleotides. In some embodiments, a spacer comprises at least 3 nucleotides. In some embodiments, a spacer comprises at most 2, 3, 4, 5, 6, 7, 8, 9, 10, 11, 12, 13, 14, 15, 16, 17, 18, 19 or 20 nucleotides. Each possibility represents a separate embodiment of the invention. In some embodiments, a spacer comprises at most 3 nucleotides. In some embodiments, a spacer comprises at most 4 nucleotides. In some embodiments, a spacer comprises at most 6 nucleotides. In some embodiments, a spacer comprises or consists of between 1-20, 1-15, 1-10, 1-9, 1-8, 1-7, 1-6, 1-5, 1-4, 1-3, 2-20, 2-15, 2-10, 2-9, 2-8, 2-7, 2-6, 2-5, 2-4, 2-3, 3-20, 3-15, 3-10, 3-9, 3-8, 3-7, 3-6, 3-5, or 3-4 nucleotides. Each possibility represents a separate embodiment of the invention. In some embodiments, a spacer comprises or consists of between 1-6 nucleotides. In some embodiments, a spacer comprises or consists of between 1-3 nucleotides. In some embodiments, a spacer comprises or consists of between 2-6 nucleotides. In some embodiments, a spacer comprises or consists of between 2-3 nucleotides. In some embodiments, a spacer comprises or consists of between 3-6 nucleotides. In some embodiments, a spacer comprises or consists of three nucleotides.

In some embodiments, the molecule further comprises a 5′ terminal spacer. In some embodiments, the molecule further comprises a 3′ terminal spacer. As used herein, the term “terminal spacer” refers to nucleotides at either end of the molecule which are neither a binding site nor an enhancer motif. Terminal spacers ensure that the binding site and/or enhancer motifs are internal to the molecule and not at an end. In some embodiments, a terminal spacer is the same length as a spacer. In some embodiments, the terminal spacer comprises or consists of two nucleotides. In some embodiments, the 3′ terminal spacer is GAG.

In some embodiments, the molecule comprises a plurality of binding sites and at least two of the binding sites are separated by a third spacer. In some embodiments, all binding sites are separated by a spacer. In some embodiments, each motif if separated by a spacer. In some embodiments, all binding sites and motifs are separated from each other by spacers.

In some embodiments, the molecule binds to YTHDF2. In some embodiments, the molecule binds to YTHDF2 more strongly that a naturally occurring RNA. In some embodiments, a naturally occurring RNA is all naturally occurring RNAs. In some embodiments, RNA is mRNA. In some embodiments, the molecule comprises increased binding to YTHDF2. In some embodiments, increased is as compared to a naturally occurring RNA. In some embodiments, increased is as compared to a control decoy RNA. In some embodiments, a control decoy RNA is a decoy RNA known in the art. In some embodiments, a decoy RNA known in the art is an RNA disclosed in Dominissini et al., 2012. In some embodiments, a decoy RNA known in the art consists of SEQ ID NO: 1. In some embodiments, increased is increased by at least 5, 10, 15, 20, 25, 30, 35, 40, 45, 50, 55, 60, 65, 70, 75, 80, 85, 90, 95, 100, 125, 150, 175, 200, 250, 300, 350, 400, 450, 500, 1000, 10000, or 100000%. Each possibility represents a separate embodiment of the invention. In some embodiments, increased is increased by at least 20%. In some embodiments, increased is increased by at least 50%. In some embodiments, increased is increased by at least 100%. In some embodiments, increased is increased by at least 1000%. In some embodiments, increased is increased by at least 10000%. In some embodiments, increased is increased by at least one order of magnitude. In some embodiments, increased is increased by at least two orders of magnitude. In some embodiments, increased is increased by at least three orders of magnitude. In some embodiments, increased is increased by at least a factor of 10, 20, 30, 40, 50, 60, 70, 80, 90, 100, 200, 300, 400, 500, 600, 700, 800, 900, or 1000. Each possibility represents a separate embodiment of the invention. In some embodiments, increased is increased by at least a factor of 10. In some embodiments, increased is increased by at least a factor of 100. In some embodiments, increased is increased by at least a factor of 1000.

In some embodiments, the molecule comprises a nucleotide sequence of SEQ ID NO: 21. In some embodiments, the molecule comprises a nucleotide sequence consisting of SEQ ID NO: 21. In some embodiments, the sequence of the molecule consists of SEQ ID NO: 21. In some embodiments, the molecule consists of SEQ ID NO: 21. In some embodiments, the molecule comprises a nucleotide sequence of SEQ ID NO: 22. In some embodiments, the molecule comprises a nucleotide sequence consisting of SEQ ID NO: 22. In some embodiments, the sequence of the molecule consists of SEQ ID NO: 22. In some embodiments, the molecule consists of SEQ ID NO: 22. In some embodiments, the molecule comprises a nucleotide sequence of SEQ ID NO: 23. In some embodiments, the molecule comprises a nucleotide sequence consisting of SEQ ID NO: 23. In some embodiments, the sequence of the molecule consists of SEQ ID NO: 23. In some embodiments, the molecule consists of SEQ ID NO: 23. In some embodiments, the molecule comprises a nucleotide sequence of SEQ ID NO: 24. In some embodiments, the molecule comprises a nucleotide sequence consisting of SEQ ID NO: 24. In some embodiments, the sequence of the molecule consists of SEQ ID NO: 24. In some embodiments, the molecule consists of SEQ ID NO: 24. In some embodiments, the molecule comprises a nucleotide sequence of SEQ ID NO: 25. In some embodiments, the molecule comprises a nucleotide sequence consisting of SEQ ID NO: 25. In some embodiments, the sequence of the molecule consists of SEQ ID NO: 25. In some embodiments, the molecule consists of SEQ ID NO: 25. In some embodiments, the molecule comprises a nucleotide sequence of SEQ ID NO: 28. In some embodiments, the molecule comprises a nucleotide sequence consisting of SEQ ID NO: 28. In some embodiments, the sequence of the molecule consists of SEQ ID NO: 28. In some embodiments, the molecule consists of SEQ ID NO: 28. In some embodiments, the molecule comprises a nucleotide sequence of SEQ ID NO: 29. In some embodiments, the molecule comprises a nucleotide sequence consisting of SEQ ID NO: 29. In some embodiments, the sequence of the molecule consists of SEQ ID NO: 29. In some embodiments, the molecule consists of SEQ ID NO: 29. In some embodiments, the molecule comprises a nucleotide sequence of SEQ ID NO: 30. In some embodiments, the molecule comprises a nucleotide sequence consisting of SEQ ID NO: 30. In some embodiments, the sequence of the molecule consists of SEQ ID NO: 30. In some embodiments, the molecule consists of SEQ ID NO: 30. In some embodiments, the molecule comprises a nucleotide sequence of SEQ ID NO: 32. In some embodiments, the molecule comprises a nucleotide sequence consisting of SEQ ID NO: 32. In some embodiments, the sequence of the molecule consists of SEQ ID NO: 32. In some embodiments, the molecule consists of SEQ ID NO: 32. In some embodiments, the molecule comprises a nucleotide sequence of SEQ ID NO: 33. In some embodiments, the molecule comprises a nucleotide sequence consisting of SEQ ID NO: 33. In some embodiments, the sequence of the molecule consists of SEQ ID NO: 33. In some embodiments, the molecule consists of SEQ ID NO: 33. In some embodiments, the molecule comprises a nucleotide sequence of SEQ ID NO: 34. In some embodiments, the molecule comprises a nucleotide sequence consisting of SEQ ID NO: 34. In some embodiments, the sequence of the molecule consists of SEQ ID NO: 34. In some embodiments, the molecule consists of SEQ ID NO: 34. In some embodiments, the molecule comprises a nucleotide sequence of SEQ ID NO: 35. In some embodiments, the molecule comprises a nucleotide sequence consisting of SEQ ID NO: 35. In some embodiments, the sequence of the molecule consists of SEQ ID NO: 35. In some embodiments, the molecule consists of SEQ ID NO: 35. In some embodiments, the molecule comprises a nucleotide sequence of SEQ ID NO: 36. In some embodiments, the molecule comprises a nucleotide sequence consisting of SEQ ID NO: 36. In some embodiments, the sequence of the molecule consists of SEQ ID NO: 36. In some embodiments, the molecule consists of SEQ ID NO: 36. In some embodiments, the molecule comprises a nucleotide sequence of SEQ ID NO: 37. In some embodiments, the molecule comprises a nucleotide sequence consisting of SEQ ID NO: 37. In some embodiments, the sequence of the molecule consists of SEQ ID NO: 37. In some embodiments, the molecule consists of SEQ ID NO: 37. In some embodiments, the molecule comprises a nucleotide sequence consisting of SEQ ID NO: 38. In some embodiments, the sequence of the molecule consists of SEQ ID NO: 38. In some embodiments, the molecule consists of SEQ ID NO: 38. In some embodiments, the molecule comprises a nucleotide sequence consisting of SEQ ID NO: 39. In some embodiments, the sequence of the molecule consists of SEQ ID NO: 379. In some embodiments, the molecule consists of SEQ ID NO: 39. In some embodiments, the molecule comprises a nucleotide sequence consisting of SEQ ID NO: 40. In some embodiments, the sequence of the molecule consists of SEQ ID NO: 40. In some embodiments, the molecule consists of SEQ ID NO: 40. In some embodiments, the molecule comprises a nucleotide sequence consisting of SEQ ID NO: 41. In some embodiments, the sequence of the molecule consists of SEQ ID NO: 41. In some embodiments, the molecule consists of SEQ ID NO: 41. In some embodiments, the molecule comprises a nucleotide sequence consisting of SEQ ID NO: 42. In some embodiments, the sequence of the molecule consists of SEQ ID NO: 42. In some embodiments, the molecule consists of SEQ ID NO: 42. In some embodiments, the molecule comprises a sequence selected from SEQ ID NO: 21-25, 28-30 and 32-37. In some embodiments, the molecule comprises a sequence selected from SEQ ID NO: 21-25, and 32-37. In some embodiments, the sequence of the molecule consists of a sequence selected from SEQ ID NO: 21-25, 28-30 and 32-37. In some embodiments, the sequence of the molecule consists of a sequence selected from SEQ ID NO: 21-25, and 32-37. In some embodiments, the molecule comprises a sequence selected from SEQ ID NO: 21-25, 28-30 and 32-42. In some embodiments, the molecule comprises a sequence selected from SEQ ID NO: 21-25, and 32-42. In some embodiments, the sequence of the molecule consists of a sequence selected from SEQ ID NO: 21-25, 28-30 and 32-42. In some embodiments, the sequence of the molecule consists of a sequence selected from SEQ ID NO: 21-25, and 32-42.

In some embodiments, the molecule comprises a nucleotide sequence consisting of SEQ ID NO: 65. In some embodiments, the sequence of the molecule consists of SEQ ID NO: 65. In some embodiments, the molecule consists of SEQ ID NO: 65. In some embodiments, the molecule comprises a nucleotide sequence consisting of SEQ ID NO: 66. In some embodiments, the sequence of the molecule consists of SEQ ID NO: 66. In some embodiments, the molecule consists of SEQ ID NO: 66. In some embodiments, the molecule comprises a sequence selected from SEQ ID NO: 21-25, 28-30, 32-42 and 65-66. In some embodiments, the molecule comprises a sequence selected from SEQ ID NO: 21-25, 32-42 and 65-66. In some embodiments, the sequence of the molecule consists of a sequence selected from SEQ ID NO: 21-25, 28-30, 32-42 and 65-66. In some embodiments, the sequence of the molecule consists of a sequence selected from SEQ ID NO: 21-25, 32-42 and 65-66.

In some embodiments, the molecule comprises a 5′ spacer comprising at least 2 2′O methylated RNA bases, the 5′ enhancer motif GGGAAACU in which all bases are 2′O methylated RNA bases, the binding site GGACT wherein the adenosine is a N6 methyladenosine and all bases are DNA bases, and a 3′ spacer comprising at least 3 2′O methylated RNA bases. In some embodiments, the molecule comprises from 5′ to 3′ in order a 5′ spacer comprising at least 2 2′O methylated RNA bases, the 5′ enhancer motif GGGAAACU in which all bases are 2′O methylated RNA bases, the binding site GGACT wherein the adenosine is a N6 methyladenosine and all bases are DNA bases, and a 3′ spacer comprising at least 3 2′O methylated RNA bases. In some embodiments, the molecule consists of a 5′ spacer comprising at least 2 2′O methylated RNA bases, the 5′ enhancer motif GGGAAACU in which all bases are 2′O methylated RNA bases, the binding site GGACT wherein the adenosine is a N6 methyladenosine and all bases are DNA bases, and a 3′ spacer comprising at least 3 2′O methylated RNA bases. In some embodiments, the molecule consists of from 5′ to 3′ in order a 5′ spacer comprising at least 2 2′O methylated RNA bases, the 5′ enhancer motif GGGAAACU in which all bases are 2′O methylated RNA bases, the binding site GGACT wherein the adenosine is a N6 methyladenosine and all bases are DNA bases, and a 3′ spacer comprising at least 3 2′O methylated RNA bases. In some embodiments, the molecule consists of a 5′ spacer consisting of 2 2′O methylated RNA bases, the 5′ enhancer motif GGGAAACU in which all bases are 2′O methylated RNA bases, the binding site GGACT wherein the adenosine is a N6 methyladenosine and all bases are DNA bases, and a 3′ spacer consisting of 3 2′O methylated RNA bases. In some embodiments, the molecule consists of from 5′ to 3′ in order a 5′ spacer consisting of 2 2′O methylated RNA bases, the 5′ enhancer motif GGGAAACU in which all bases are 2′O methylated RNA bases, the binding site GGACT wherein the adenosine is a N6 methyladenosine and all bases are DNA bases, and a 3′ spacer consisting of 3 2′O methylated RNA bases. In some embodiments, the molecule comprises SEQ ID NO: 67. In some embodiments, the molecule consists of SEQ ID NO: 67. In some embodiments, the molecule is SEQ ID NO: 67.

In some embodiments, the molecule comprises a 5′ spacer comprising at least 2 2′O methylated RNA bases, the 5′ enhancer motif GGGAAACU in which all bases are RNA bases and some bases are 2′O methylated, the binding site GGACT wherein the adenosine is a N6 methyladenosine and all bases are DNA bases, and a 3′ spacer comprising at least 3 2′O methylated RNA bases. In some embodiments, the molecule comprises from 5′ to 3′ in order a 5′ spacer comprising at least 2 2′O methylated RNA bases, the 5′ enhancer motif GGGAAACU in which all bases are RNA bases and some bases are 2′O methylated, the binding site GGACT wherein the adenosine is a N6 methyladenosine and all bases are DNA bases, and a 3′ spacer comprising at least 3 2′O methylated RNA bases. In some embodiments, the molecule consists of a 5′ spacer comprising at least 2 2′O methylated RNA bases, the 5′ enhancer motif GGGAAACU in which all bases are RNA bases and some bases are 2′O methylated, the binding site GGACT wherein the adenosine is a N6 methyladenosine and all bases are DNA bases, and a 3′ spacer comprising at least 3 2′O methylated RNA bases. In some embodiments, the molecule consists of from 5′ to 3′ in order a 5′ spacer comprising at least 2 2′O methylated RNA bases, the 5′ enhancer motif GGGAAACU in which all bases are RNA bases and some bases are 2′O methylated, the binding site GGACT wherein the adenosine is a N6 methyladenosine and all bases are DNA bases, and a 3′ spacer comprising at least 3 2′O methylated RNA bases. In some embodiments, the molecule consists of a 5′ spacer consisting of 2 2′O methylated RNA bases, the 5′ enhancer motif GGGAAACU in which all bases are RNA bases and some bases are 2′O methylated, the binding site GGACT wherein the adenosine is a N6 methyladenosine and all bases are DNA bases, and a 3′ spacer consisting of 3 2′O methylated RNA bases. In some embodiments, the molecule consists of from 5′ to 3′ in order a 5′ spacer consisting of 2 2′O methylated RNA bases, the 5′ enhancer motif GGGAAACU in which all bases are RNA bases and some bases are 2′O methylated, the binding site GGACT wherein the adenosine is a N6 methyladenosine and all bases are DNA bases, and a 3′ spacer consisting of 3 2′O methylated RNA bases. In some embodiments, some bases is at least 1 base. In some embodiments, some bases is at least 2 bases. In some embodiments, some bases is at least 3 bases. In some embodiments, some bases is at least 4 bases. In some embodiments, the first G of GGGAAACU is 2′O methylated. In some embodiments, the last A of GGGAAACU is 2′O methylated. In some embodiments, the C of GGGAAACU is 2′O methylated. In some embodiments, the U of GGGAAACU is 2′O methylated. In some embodiments, the molecule comprises SEQ ID NO: 68. In some embodiments, the molecule consists of SEQ ID NO: 68. In some embodiments, the molecule is SEQ ID NO: 68.

In some embodiments, the molecule comprises SEQ ID NO: 69. In some embodiments, the molecule consists of SEQ ID NO: 69. In some embodiments, the molecule is SEQ ID NO: 69.

In some embodiments, the molecule comprises a nucleotide sequence of SEQ ID NO: 1. In some embodiments, the molecule comprises a nucleotide sequence consisting of SEQ ID NO: 1. In some embodiments, the sequence of the molecule consists of SEQ ID NO: 1. In some embodiments, the molecule consists of SEQ ID NO: 1. In some embodiments, the molecule comprises a nucleotide sequence of SEQ ID NO: 2. In some embodiments, the molecule comprises a nucleotide sequence consisting of SEQ ID NO: 2. In some embodiments, the sequence of the molecule consists of SEQ ID NO: 2. In some embodiments, the molecule consists of SEQ ID NO: 2. In some embodiments, the molecule comprises a nucleotide sequence of SEQ ID NO: 3. In some embodiments, the molecule comprises a nucleotide sequence consisting of SEQ ID NO: 3. In some embodiments, the sequence of the molecule consists of SEQ ID NO: 3. In some embodiments, the molecule consists of SEQ ID NO: 3. In some embodiments, the molecule comprises a nucleotide sequence of SEQ ID NO: 4. In some embodiments, the molecule comprises a nucleotide sequence consisting of SEQ ID NO: 4. In some embodiments, the sequence of the molecule consists of SEQ ID NO: 4. In some embodiments, the molecule consists of SEQ ID NO: 4. In some embodiments, the molecule comprises a nucleotide sequence of SEQ ID NO: 5. In some embodiments, the molecule comprises a nucleotide sequence consisting of SEQ ID NO: 5. In some embodiments, the sequence of the molecule consists of SEQ ID NO: 5. In some embodiments, the molecule consists of SEQ ID NO: 5. In some embodiments, the molecule comprises a nucleotide sequence of SEQ ID NO: 6. In some embodiments, the molecule comprises a nucleotide sequence consisting of SEQ ID NO: 6. In some embodiments, the sequence of the molecule consists of SEQ ID NO: 6. In some embodiments, the molecule consists of SEQ ID NO: 6. In some embodiments, the molecule comprises a nucleotide sequence of SEQ ID NO: 7. In some embodiments, the molecule comprises a nucleotide sequence consisting of SEQ ID NO: 7. In some embodiments, the sequence of the molecule consists of SEQ ID NO: 7. In some embodiments, the molecule consists of SEQ ID NO: 7. In some embodiments, the molecule comprises a nucleotide sequence of SEQ ID NO: 8. In some embodiments, the molecule comprises a nucleotide sequence consisting of SEQ ID NO: 8. In some embodiments, the sequence of the molecule consists of SEQ ID NO: 8. In some embodiments, the molecule consists of SEQ ID NO: 8. In some embodiments, the molecule comprises a nucleotide sequence of SEQ ID NO: 9. In some embodiments, the molecule comprises a nucleotide sequence consisting of SEQ ID NO: 9. In some embodiments, the sequence of the molecule consists of SEQ ID NO: 9. In some embodiments, the molecule consists of SEQ ID NO: 9. In some embodiments, the molecule comprises a nucleotide sequence of SEQ ID NO: 11. In some embodiments, the molecule comprises a nucleotide sequence consisting of SEQ ID NO: 11 In some embodiments, the sequence of the molecule consists of SEQ ID NO: 11. In some embodiments, the molecule consists of SEQ ID NO: 11. In some embodiments, the molecule comprises a nucleotide sequence of SEQ ID NO: 12. In some embodiments, the molecule comprises a nucleotide sequence consisting of SEQ ID NO: 12. In some embodiments, the sequence of the molecule consists of SEQ ID NO: 12. In some embodiments, the molecule consists of SEQ ID NO: 12. In some embodiments, the molecule comprises a nucleotide sequence of SEQ ID NO: 13. In some embodiments, the molecule comprises a nucleotide sequence consisting of SEQ ID NO: 13. In some embodiments, the sequence of the molecule consists of SEQ ID NO: 13. In some embodiments, the molecule consists of SEQ ID NO: 13. In some embodiments, the molecule comprises a nucleotide sequence of SEQ ID NO: 14. In some embodiments, the molecule comprises a nucleotide sequence consisting of SEQ ID NO:

14. In some embodiments, the sequence of the molecule consists of SEQ ID NO: 14. In some embodiments, the molecule consists of SEQ ID NO: 14. In some embodiments, the molecule comprises a nucleotide sequence of SEQ ID NO: 15. In some embodiments, the molecule comprises a nucleotide sequence consisting of SEQ ID NO: 15. In some embodiments, the sequence of the molecule consists of SEQ ID NO: 15. In some embodiments, the molecule consists of SEQ ID NO: 15. In some embodiments, the molecule comprises a nucleotide sequence of SEQ ID NO: 16. In some embodiments, the molecule comprises a nucleotide sequence consisting of SEQ ID NO: 16. In some embodiments, the sequence of the molecule consists of SEQ ID NO: 16. In some embodiments, the molecule consists of SEQ ID NO: 16. In some embodiments, the molecule comprises a nucleotide sequence of SEQ ID NO: 17. In some embodiments, the molecule comprises a nucleotide sequence consisting of SEQ ID NO: 17. In some embodiments, the sequence of the molecule consists of SEQ ID NO: 17. In some embodiments, the molecule consists of SEQ ID NO: 17. In some embodiments, the molecule comprises a nucleotide sequence of SEQ ID NO: 18. In some embodiments, the molecule comprises a nucleotide sequence consisting of SEQ ID NO: 18. In some embodiments, the sequence of the molecule consists of SEQ ID NO: 18. In some embodiments, the molecule consists of SEQ ID NO: 18. In some embodiments, the molecule comprises a nucleotide sequence of SEQ ID NO: 19. In some embodiments, the molecule comprises a nucleotide sequence consisting of SEQ ID NO: 19. In some embodiments, the sequence of the molecule consists of SEQ ID NO: 19. In some embodiments, the molecule consists of SEQ ID NO: 19. In some embodiments, the molecule comprises a nucleotide sequence of SEQ ID NO: 20. In some embodiments, the molecule comprises a nucleotide sequence consisting of SEQ ID NO: 20. In some embodiments, the sequence of the molecule consists of SEQ ID NO: 20. In some embodiments, the molecule consists of SEQ ID NO: 20. In some embodiments, the molecule comprises a nucleotide sequence of SEQ ID NO: 26. In some embodiments, the molecule comprises a nucleotide sequence consisting of SEQ ID NO: 26. In some embodiments, the sequence of the molecule consists of SEQ ID NO: 26. In some embodiments, the molecule consists of SEQ ID NO: 26. In some embodiments, the molecule comprises a nucleotide sequence of SEQ ID NO: 27. In some embodiments, the molecule comprises a nucleotide sequence consisting of SEQ ID NO: 27. In some embodiments, the sequence of the molecule consists of SEQ ID NO: 27. In some embodiments, the molecule consists of SEQ ID NO: 27. In some embodiments, the molecule comprises a nucleotide sequence of SEQ ID NO: 31. In some embodiments, the molecule comprises a nucleotide sequence consisting of SEQ ID NO: 31. In some embodiments, the sequence of the molecule consists of SEQ ID NO: 31. In some embodiments, the molecule consists of SEQ ID NO: 31. In some embodiments, the molecule comprises a nucleotide sequence of SEQ ID NO: 51. In some embodiments, the molecule comprises a nucleotide sequence consisting of SEQ ID NO: 51. In some embodiments, the sequence of the molecule consists of SEQ ID NO: 51. In some embodiments, the molecule consists of SEQ ID NO: 51. In some embodiments, the molecule comprises a nucleotide sequence of SEQ ID NO: 52. In some embodiments, the molecule comprises a nucleotide sequence consisting of SEQ ID NO: 52. In some embodiments, the sequence of the molecule consists of SEQ ID NO: 52. In some embodiments, the molecule consists of SEQ ID NO: 52. In some embodiments, the molecule comprises a nucleotide sequence of SEQ ID NO: 53. In some embodiments, the molecule comprises a nucleotide sequence consisting of SEQ ID NO: 53. In some embodiments, the sequence of the molecule consists of SEQ ID NO: 53. In some embodiments, the molecule consists of SEQ ID NO: 53. In some embodiments, the molecule comprises a nucleotide sequence of SEQ ID NO: 54. In some embodiments, the molecule comprises a nucleotide sequence consisting of SEQ ID NO: 54. In some embodiments, the sequence of the molecule consists of SEQ ID NO: 54. In some embodiments, the molecule consists of SEQ ID NO: 54. In some embodiments, the molecule comprises a nucleotide sequence of SEQ ID NO: 55. In some embodiments, the molecule comprises a nucleotide sequence consisting of SEQ ID NO: 55. In some embodiments, the sequence of the molecule consists of SEQ ID NO: 55. In some embodiments, the molecule consists of SEQ ID NO: 55. In some embodiments, the molecule comprises a nucleotide sequence of SEQ ID NO: 65. In some embodiments, the molecule comprises a nucleotide sequence consisting of SEQ ID NO: 65. In some embodiments, the sequence of the molecule consists of SEQ ID NO: 65. In some embodiments, the molecule consists of SEQ ID NO: 65. In some embodiments, the molecule comprises a nucleotide sequence of SEQ ID NO: 66. In some embodiments, the molecule comprises a nucleotide sequence consisting of SEQ ID NO: 66 In some embodiments, the sequence of the molecule consists of SEQ ID NO: 66. In some embodiments, the molecule consists of SEQ ID NO: 66.

In some embodiments, the molecule comprises a sequence selected from SEQ ID NO: 1-9, 11-37 and 51-55. In some embodiments, the molecule comprises a sequence selected from SEQ ID NO: 1-3, 5-6, 8-9, 11-26, 28-37 and 51-55. In some embodiments, the sequence of the molecule consists of a sequence selected from SEQ ID NO: 1-9, 11-37 and 51-55. In some embodiments, the sequence of the molecule consists of a sequence selected from SEQ ID NO: 1-3, 5-6, 8-9, 11-26, 28-37 and 51-55. In some embodiments, the molecule comprises a sequence selected from SEQ ID NO: 1-9, 11-42, 51-55 and 65-66. In some embodiments, the molecule comprises a sequence selected from SEQ ID NO: 1-3, 5-6, 8-9, 11-26, 28-42, 51-55 and 66. In some embodiments, the sequence of the molecule consists of a sequence selected from SEQ ID NO: 1-9, 11-42, 51-55 and 65-66. In some embodiments, the sequence of the molecule consists of a sequence selected from SEQ ID NO: 1-3, 5-6, 8-9, 11-26, 28-43, 51-55 and 66.

By another aspect, there is provided a composition comprising a nucleic acid molecule of the invention.

In some embodiments, the composition is a pharmaceutical composition. In some embodiments, the composition is a therapeutic composition. In some embodiments, the composition is a diagnostic composition. In some embodiments, the composition is formulated for administration to a subject. In some embodiments, the composition is formulated for systemic administration. In some embodiments, the composition is formulated for local administration. In some embodiments, local is local to a site of a disease or condition. In some embodiments, local administration is intratumoral administration.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. Other suitable routes of administration can include parenteral, subcutaneous, oral, intramuscular, intratumoral, or intraperitoneal.

The dosage administered will be dependent upon the age, health, and weight of the recipient, kind of concurrent treatment, if any, frequency of treatment, and the nature of the effect desired.

In some embodiments, the subject is a mammal. In some embodiments, the subject is a human. In some embodiments, the subject is need of the composition of the invention. In some embodiments, the subject is need of treatment with a composition of the invention. In some embodiments, the subject is in need of a method of the invention. In some embodiments, the subject is in need of reduction of YTHDF2 function. In some embodiments, the subject is in need of YTHDF2 inhibition. In some embodiments, the subject suffers from cancer. In some embodiments, the YTHDF2 function is RNA degradation. In some embodiments, RNA degradation is degradation of RNAs comprising an m6A modification. In some embodiments, the RNA is messenger RNA (mRNA). In some embodiments, the subject suffers from a disease or condition characterized or caused by YTHDF2 mediated mRNA instability. In some embodiments, the subject suffers from a disease or condition characterized or caused by YTHDF2 mediated mRNA degradation. In some embodiments, the subject suffers from cancer. In some embodiments, the subject suffers from an infection. In some embodiments, an infection is a viral infection.

In some embodiments, the subject comprises increased YTHDF2 expression. In some embodiments, a cell of the disease comprises YTHDF2 expression. In some embodiments, increased is as compared to a healthy control. In some embodiments, a healthy control is a healthy control cell. In some embodiments, the control cell is of the same cell type as the cell of the disease. In some embodiments, a cell of a disease is a disease cell. In some embodiments, a disease cell is a cancerous cell. In some embodiments, the cancerous cell comprises increased YTHDF2 expression. In some embodiments, a tumor comprises increased YTHDF2 expression. In some embodiments, the disease cell is an infected cell. In some embodiments, the disease cell is a cell infected with a virus.

In some embodiments, the composition further comprises a pharmaceutically acceptable carrier, excipient or adjuvant. In some embodiments, the composition comprises a therapeutically effective amount of the nucleic acid molecule of the invention. A therapeutically effective amount of the nucleic acid molecule is an amount sufficient to produce a therapeutic benefit. In some embodiments, effective is effective in inhibiting YTHDF2. In some embodiments, inhibiting YTHDF2 is inhibiting YTHDF2 function. In some embodiments, YTHDF2 function is mRNA degradation. In some embodiments, effective is effective to treat the cancer. In some embodiments, effective is effective to treat an infection. In some embodiments, an infection is a viral infection.

As used herein, the term “carrier,” “adjuvant” or “excipient” refers to any component of a pharmaceutical composition that is not the active agent. As used herein, the term “pharmaceutically acceptable carrier” refers to non-toxic, inert solid, semi-solid liquid filler, gel, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

By another aspect, there is provided a method of inhibiting YTHDF2, the method comprising contacting the YTHDF2 with a nucleic acid molecule of the invention or a composition of the invention, thereby inhibiting YTHDF2.

In some embodiments, the composition is for use in inhibiting YTHDF2. In some embodiments, inhibiting YTHDF2 is inhibiting YTHDF2 in a cell. In some embodiments, the method comprises contacting the cell with a nucleic acid molecule of the invention or a composition of the invention. In some embodiments, the contacting comprises delivering the nucleic acid molecule into the cytosol of the cell. In some embodiments, inhibiting is inhibiting YTHDF2 function. In some embodiments, YTHDF2 function is binding. In some embodiments, binding is binding to RNA. In some embodiments, binding is binding to RNA comprising an m6A. In some embodiments, inhibiting is inhibiting YTHDF2 mediated degradation of RNA.

In some embodiments, inhibiting is inhibiting YTHDF2 mediated degradation of RNA comprising an m6A. In some embodiments, the RNA is mRNA.

Methods and assays for measuring binding and mRNA stability and degradation are well known in the art. Several such methods are provided hereinbelow. Any known method may be used for determining binding (e.g., an ELISA) and/or for determining mRNA degradation (e.g., measuring levels of the RNA before and after addition of the nucleic acid molecule).

In some embodiments, inhibiting comprises an inhibition or decrease of at least 5, 10, 15, 20, 25, 30, 35, 40, 45, 50, 55, 60, 65, 70, 75, 80, 85, 90, 95, or 100%. Each possibility represents a separate embodiment of the invention. In some embodiments, inhibiting comprises an inhibition or decrease of at least 20%. In some embodiments, inhibiting comprises an inhibition or decrease of at least 50%. In some embodiments, inhibiting comprises an inhibition or decrease of at least 90%.

By another aspect, there is provided a method of treating a disease or condition characterized by YTHDF2 mediated mRNA instability or degradation in a subject in need thereof, the method comprising administering to the subject a nucleic acid molecule of the invention or a composition of the invention, thereby treating a disease or condition.

By another aspect, there is provided a method of treating cancer in a subject in need thereof, the method comprising administering to the subject a nucleic acid molecule of the invention or a composition of the invention, thereby treating cancer.

In some embodiments, the composition is for use in treating a disease or condition characterized by YTHDF2 mediated mRNA instability or degradation. In some embodiments, the composition is for use in treating cancer. In some embodiments, the disease is characterized by mRNA instability. In some embodiments, YTHDF2 mediated RNA instability or degradation causes the disease or condition. In some embodiments, YTHDF2 mediated RNA instability or degradation sustains the disease or condition. In some embodiments, YTHDF2 mediated RNA instability or degradation enhances the disease or condition. In some embodiments, the instability is of an mRNA of a disease-associated gene. In some embodiments, the instability is of a disease-associated mRNA. In some embodiments, disease-associated is disease-causing. In some embodiments, the mRNA is a disease-associated mRNA. In some embodiments, the mRNA is a disease-causing mRNA. In some embodiments, the mRNA is of a gene. In some embodiments, the gene is a disease-associated gene. In some embodiments, the gene is a disease-causing gene.

As used herein, a “disease-associated gene” is a gene whose function, or loss of function contributes to the disease. In some embodiments, the gene is a protein coding gene. In some embodiments, decreased expression of the gene is associated with the disease. In some embodiments, decreased expression of the gene causes the disease. In some embodiments, decreased expression is loss of expression. In some embodiments, decreased function of the protein encoded by the gene is associated with the disease. In some embodiments, decreased function of the protein encoded by the gene causes the disease. In some embodiments, decreased expression is increased degradation. In some embodiments, degradation is degradation of the mRNA. In some embodiments, increased degradation comprises a shorter half-life. In some embodiments, half-life is mRNA half-life within a cell. In some embodiments, the disease-associated gene is a tumor suppressor gene. Examples of genes whose loss/decrease in expression or function are associated with a disease include, but are not limited to TNFRSF1B, PRSS23, ID2, DLK2, DAPK2, AXIN1, YAP, LHPP, MOB3B, and NKX3-1. Methods of determining mRNA instability in a sample or a subject are well known in the art and any method may be used. Methods of determining mRNA stability or degradation include, but are not limited to, PCR, norther blots, pulse chase experiments, radioactive labeling, and reporter assays.

In some embodiments, mRNA instability comprises aberrant mRNA degradation. In some embodiments, mRNA degradation is measured by measuring steady state mRNA levels. In some embodiments, mRNA stability is mRNA half-life. In some embodiments, half-life is half-life in a cell. In some embodiments, increased mRNA degradation comprises a decreased mRNA half-life. In some embodiments, aberrant mRNA degradation comprises mRNA half-life below a predetermined threshold. In some embodiments, the threshold is the mRNA's half-life in a healthy control. In some embodiments, the healthy control is a healthy subject. In some embodiments, the healthy control is a healthy cell. In some embodiments, healthy is non-diseased. In some embodiments, mRNA degradation is measured by measuring mRNA levels. In some embodiments, mRNA transcription is inhibited by actinomycin. In some embodiments, mRNA instability is confirmed by treatment with actinomycin and measuring mRNA levels. In some embodiments, actinomycin is actinomycin D. In some embodiments, aberrant is increased. In some embodiments, increased is increased by at least 5, 10, 15, 20, 25, 30, 35, 40, 45, 50, 55, 60, 65, 70, 75, 80, 85, 90, 95, 100, 125, 150,175, 200, 250, 300, 350, 400, 450 or 500%. Each possibility represents a separate embodiment of the invention. In some embodiments, increased is increased by at least 20%. In some embodiments, increased is increased by at least 50%. In some embodiments, increased is increased by at least 100%. Methods of determining mRNA stability/instability are well known in the art and can be found for example in Lugowski et al., “Determining mRNA half-life on a transcriptome-wide scale”, Methods, 2018 March 15; 137:90-98, and Ratnadiwakara and Anko, “mRNA stability assay using transcription inhibition by actinomycin D in mouse pluripotent stem cells”, Bio Protoc. 2018 Nov. 5; 8 (21): e3072, both of which are hereby incorporated by reference in their entirety.

In some embodiments, the disease or condition is cancer. As used herein “cancer” or “pre-malignancy” are diseases associated with cell proliferation. Non-limiting types of cancer include carcinoma, sarcoma, lymphoma, leukemia, blastoma and germ cells tumors. In one embodiment, carcinoma refers to tumors derived from epithelial cells including but not limited to breast cancer, prostate cancer, lung cancer, pancreas cancer, and colon cancer. In one embodiment, sarcoma refers of tumors derived from mesenchymal cells including but not limited to sarcoma botryoides, chondrosarcoma, Ewing's sarcoma, malignant hemangioendothelioma, malignant schwannoma, osteosarcoma and soft tissue sarcomas. In one embodiment, lymphoma refers to tumors derived from hematopoietic cells that leave the bone marrow and tend to mature in the lymph nodes including but not limited to hodgkin lymphoma, non-hodgkin lymphoma, multiple myeloma and immunoproliferative diseases. In one embodiment, leukemia refers to tumors derived from hematopoietic cells that leave the bone marrow and tend to mature in the blood including but not limited to acute lymphoblastic leukemia, chronic lymphocytic leukemia, acute myelogenous leukemia, chronic myelogenous leukemia, hairy cell leukemia, T-cell prolymphocytic leukemia, large granular lymphocytic leukemia and adult T-cell leukemia. In one embodiment, blastoma refers to tumors derived from immature precursor cells or embryonic tissue including but not limited to hepatoblastoma, medulloblastoma, nephroblastoma, neuroblastoma, pancreatoblastoma, pleuropulmonary blastoma, retinoblastoma and glioblastoma-multiforme. In one embodiment, germ cell tumors refers to tumors derived from germ cells including but not limited to germinomatous or seminomatous germ cell tumors (GGCT, SGCT) and nongerminomatous or nonseminomatous germ cell tumors (NGGCT, NSGCT). In one embodiment, germinomatous or seminomatous tumors include but not limited to germinoma, dysgerminoma and seminoma. In one embodiment, nongerminomatous or nonseminomatous tumors refers to pure and mixed germ cells tumors including but not limited to embryonal carcinoma, endodermal sinus tumor, choriocarcinoma, tearoom, polyembryoma, gonadoblastoma and teratocarcinoma. In some embodiments, the cancer is selected from lung cancer, liver cancer, leukemia, bladder cancer, pancreatic cancer, prostate cancer and breast cancer. In some embodiments, the cancer is selected from lung cancer, liver cancer, brain cancer, renal cancer, muscle cancer, uterine cancer, cervical cancer, head and neck cancer, pancreatic cancer and breast cancer. In some embodiments, the cancer is a solid cancer. In some embodiments, the cancer is breast cancer. In some embodiments, the breast cancer is triple negative breast cancer. In some embodiments, triple negative is negative for estrogen receptor (ER), progesterone receptor (PR) and HER2. In some embodiments, the cancer is lung cancer. In some embodiments, the lung cancer is lung adenocarcinoma. In some embodiments, the lung cancer is lung carcinoma. In some embodiments, the cancer is liver cancer. In some embodiments, the liver cancer is hepatocellular carcinoma (HCC). In some embodiments, the cancer is an epithelial cancer. In some embodiments, the cancer is a carcinoma. In some embodiments, the carcinoma is adenocarcinoma. In some embodiments, the carcinoma is ductal carcinoma. In some embodiments, the cancer is pancreatic cancer. In some embodiments, the cancer is head and neck cancer. In some embodiments, the head and neck cancer is tongue cancer. In some embodiments, the cancer is tongue cancer. In some embodiments, the carcinoma is squamous cell carcinoma. In some embodiments, the cancer is brain cancer. In some embodiments, the brain cancer is glioblastoma. In some embodiments, the brain cancer is cerebellar cancer. In some embodiments, the brain cancer is desmoplastic cerebellar medulloblastoma. In some embodiments, the cancer is medulloblastoma. In some embodiments, the brain cancer is neuroblastoma. In some embodiments, the cancer is uterine cancer. In some embodiments, the cancer is cervical cancer. In some embodiments, the cancer is muscle cancer. In some embodiments, the muscle cancer is rhabdomyosarcoma. In some embodiments, the cancer is kidney cancer. In some embodiments, kidney cancer is renal cancer. In some embodiments, the cancer is a blood cancer. In some embodiments, the cancer is a hematological cancer. In some embodiments, a cell of the cancer comprises increased expression of YTHDF2 as compared to a non-cancerous cell of the same cell type. In some embodiments, the same cell type is the same tissue. In some embodiments, non-cancerous is healthy.

In some embodiments, the disease or condition is an infection. In some embodiments, the infection is a viral infection. In some embodiments, the viral infection is selected from Kaposi's Sarcoma-associated Herpesvirus (KSHV), Human Cytomegalovirus (HCMV), Human Immunodeficiency Virus 1 (HIV-1) and influenza. In some embodiments, a cell infected with the virus comprises increased YTHDF2 expression as compared to a non-infected cell.

In some embodiments, the method further comprises measuring YTHDF2 levels in a sample from the subject. In some embodiments, the method further comprises measuring RNA instability or degradation in a sample from the subject. In some embodiments, the sample comprises cells. In some embodiments, the cells are disease cells. In some embodiments, the method further comprises measuring YTHDF2 levels in a control sample from the subject. In some embodiments, the method further comprises measuring RNA instability or degradation in a control sample from the subject. In some embodiments, the cells are healthy cells. In some embodiments, the presence of YTHDF2 levels above a predetermined threshold indicates the subject is suitable to be treated by a method of the invention. In some embodiments, the presence of RNA instability or degradation above a predetermined threshold indicates the subject is suitable to be treated by a method of the invention. In some embodiments, the RNA instability is YTHDF2 mediated instability. In some embodiments, the RNA degradation is YTHDF2 mediated degradation. In some embodiments, presence is presence in the sample. In some embodiments, presence is presence in the disease cells. In some embodiments, the predetermined threshold is the levels in the control sample. In some embodiments, the predetermined threshold is the levels in the control cells of the control sample. In some embodiments, the predetermined threshold is the levels in control cells. In some embodiments, control cells are non-disease cells of the same cell type as the disease cells. In some embodiments, cell type is tissue.

By another aspect, there is provided a method of determining suitability of a subject to be treated by a method of the invention, the method comprising measuring YTHDF2 levels or mRNA instability or degradation is a sample from the subject, wherein the presence of YTHDF2 levels or mRNA instability or degradation above a predetermined threshold indicates the subject is suitable to be treated by a method of the invention.

In some embodiments, the method is a diagnostic method. In some embodiments, the method is an ex vivo method. In some embodiments, the method is an in vitro method. In some embodiments, the method further comprises receiving the disease sample. In some embodiments, the method further comprises obtaining the disease sample. In some embodiments, the method further comprises extracting a sample comprising disease cells from the subject. In some embodiments, the method further comprises receiving the control sample. In some embodiments, the method further comprises obtaining the control sample. In some embodiments, the method further comprises extracting a sample comprising control cells from the subject. In some embodiments, the sample is a disease sample. In some embodiments, the sample is a control sample. In some embodiments, the method further comprises administering the nucleic acid molecule of the invention or the composition of the invention to a subject determined to be suitable. In some embodiments, the method further comprises administering an alternative therapeutic to a subject determined to be unsuitable.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Materials and Methods

Cell Culture conditions: Cell line specific culture conditions are provided in Table 10.

TABLE 10
Cell culture conditions

| Cell line | Base medium | Additives |
|---|---|---|
| A375 | DMEM | 10% FCS; 1% PSA; 1% L-Glu; |
| | | 1% sodium pyruvate |
| A-673 | DMEM | 10% FCS; 1% PSA; 1% L-Glu; |
| | | 1% sodium pyruvate |
| Panc-1 | DMEM | 10% FCS; 1% PSA; 1% L-Glu; |
| | | 1% sodium pyruvate |
| MIA-PaCa-2 | DMEM | 10% FCS; 1% PSA; 1% L-Glu; |
| | | 1% sodium pyruvate |
| Cal27 | DMEM | 10% FCS; 1% PSA; 1% L-Glu; |
| | | 1% sodium pyruvate |
| U87 | EMEM | 10% FCS; 1% PSA; 1% sodium |
| | | pyruvate; 0.1 mM NEAA |
| DAOY | EMEM | 10% FCS; 1% PSA; 1% sodium |
| | | pyruvate; 0.1 mM NEAA |
| SiHa | EMEM | 10% FCS; 1% PSA; 1% sodium |
| | | pyruvate; 0.1 mM NEAA |
| Hela | EMEM | 10% FCS; 1% PSA; 1% sodium |
| | | pyruvate; 0.1 mM NEAA |
| A2058 | EMEM | 10% FCS; 1% PSA; 1% sodium |
| | | pyruvate; 0.1 mM NEAA |
| RMS13 | RPMI-1640 | 10% FCS; 1% PSA; 1% L-Glu; |
| | | 1% sodium pyruvate |
| 22Rv1 | RPMI-1640 | 10% FCS; 1% PSA; 1% L-Glu; |
| | | 1% sodium pyruvate |
| 769-P | RPMI-1640 | 10% FCS; 1% PSA; 1% L-Glu; |
| | | 1% sodium pyruvate |
| 786-O | RPMI-1640 | 10% FCS; 1% PSA; 1% L-Glu; |
| | | 1% sodium pyruvate |
| Thp-1 | RPMI-1640 | 10% FCS; 1% PSA; 1% L-Glu; |
| | | 1% sodium pyruvate |
| Kassumi-1 | RPMI-1640 | 20% FCS; 1% PSA; 1% L-Glu; |
| | | 1% sodium pyruvate |
| A549 | DMEM | 10% FCS; 1% PSA; 1% L-Glu |
| Hep3B | EMEM | 10% FCS; 1% PSA; 1% L-glu |
| h1299 | RPMI-1640 | 10% FCS; 1% PSA; 1% L-Glu |
| HCC1937 | RPMI-1640 | 10% FCS; 1% PSA; 1% L-Glu; |
| | | 1% sodium pyruvate; 1% HEPES |
| MDA-MB-468 | DMEM | 10% FCS; 1% PSA; 1% L-Glu; |
| | | 1% sodium pyruvate |
| MDA-MB-231 | DMEM | 10% FCS; 1% PSA; 1% L-Glu; |
| | | 1% sodium pyruvate |
| MAXF | RPMI-1640 | 10% FBS, 1% Gentamycin; 1% L-glu |
| LXFA | RPMI-1640 | 10% FBS, 1% Gentamycin; 1% L-glu |
| HUH7D | DMEM | 10% FCS; 1% PSA; 1% L-Glu |

Decoy RNA Oligo Transfection and Viability assay: 2.5×10\hat{ }4 MDA-MB-231 cells were seeded in each well of a 96-well plate, a day prior to decoy oligo transfection. Different decoy oligos with different stabilizing modifications (Table 9) or scrambled controls were used. Triplicate wells were seeded for each oligo concentration that was used. The transfection was carried out with Lipofectamine RNAi-MAX (Thermo) following the manufacturer's protocol at a final volume of 50 μl. The cells, together with the transfection mixture were incubated for 6 hours, after which they were replenished with 150 μl fresh medium (final volume 200 μl). Cells were grown for 72 hours after transfection. A viability assay was carried out with CellTiter-Glo (IMBH) following the manufacturer's protocol.

hPBMCS isolation: Buffy coat was received from Shiba's hospital blood bank. hPBMCs were then isolated using Sepmate tubes (StemCell) according to manufacturer protocol. PBMCs were then moved into a flask and placed in an incubator for overnight before experimenting or freezing.

hPBMCS decoy oligo transfection: PBMCs were plated in 96 U-shape well plates 250 k cells per well. The PBMCs were transfected with the selected decoy oligos. A scrambled sequence oligos containing the same modifications were used as the negative controls. For a positive control of PBMC activation, the cells were transfected with CpG control and 353512 oligo. Triplicate wells were seeded for each oligo concentration that was used. The transfection was carried out with Lipofectamine RNAi-MAX (Thermo) following the manufacturer's protocol at final volume of 50 μl.

For IL-6 and TNF-α ELISA—the cells, together with the transfection mixture were incubated for 6 hours, after which they were replenished with 200 μl fresh medium (final volume 250 μl). Cells were grown for 24 hours after transfection, plates were then centrifuged at 300 g for 10 min and 200 μl of supernatant was collected into a new 96 well plate for IL-6 and TNF-α measurements and were stored in −20° C.

For viability assay—the cells, together with the transfection mixture were incubated for 6 hours, after which they were replenished with 150 μl fresh medium (final volume 200 μl). Cells were grown for 72 hours after transfection, 100 μl of cells from each well were moved into a new 96 well flat bottom plate, and the viability assay was carried out with CellTiter-Glo (IMBH) following the manufacturer's protocol.

ELISA of IL-6 and TNF-α: Both IL-6 and TNF-α were measured using ELISA MAX Deluxe Set Human kit (Biolegend). Maxisorp plates were coated the day prior to ELISA assay according to manufacturer protocol. The next day ELISA protocol was performed according to the manufacturer instructions. Supernatant of the samples for IL-6 and TNF-α were diluted 1:4 and 1:2 respectively. A standard curve was prepared as described by Lot-Specific Certificate of Analysis/ELISA MA Deluxe Set Protocol. Absorbance was measured in a Tecan plate reader at 450 nm and 570 nm, the absorbance at 570 nm was subtracted from the absorbance at 450 nm.

Caspase activity measurement: The day prior to experimentation, HeLa cells were plated in 96 well plates at 2500 cells per well. Cells were transfected with the selected decoy oligos. Negative control sequence oligo is Nusienrsen an approved sequence by the FDA. For positive control, the sequence is sequence 353512 from Shen et al., “Chemical modification of PS-ASO therapeutics reduces cellular protein-binding and improves the therapeutic index”, Nat Biotechnol. 2019 June; 37 (6): 640-650 (CCCAUUUCAGGAGACCUGG; SEQ ID NO: 74). Triplicate wells were seeded for each oligo concentration that was used. The transfection was carried out with Lipofectamine RNAi-MAX (Thermo) following the manufacturer's protocol at final volume of 50 μl. 5 hours post transfection Stausporine was added at a concentration of 5 μM for 3 hours as another positive control. 8 hours post transfection, Caspase 3/7 activation was measured using Caspase-Glo® 3/7 Assay (Promega) according to manufacturer protocol.

Plasma stability assay: CMT-124 of the positive control Patisiran at 0.2 μM in duplicate were incubated with plasma (WuXi DMPK labs). The reaction samples were stopped at multiple time points (0, 1, 2, 4, 6 and 24 hours). All samples were immediately mixed with stop solution containing internal standard (IS) to stop the reaction. Samples were analyzed by LC/MS/MS, and the disappearance of test compound was assessed based on peak area ratios of analyte/IS (no standard curve).

Liver S9 Metabolic stability assay: CMT-124 of the positive control Patisiran in duplicate were incubated at 37° C. with liver S9 (pooled from multiple donors) at 1.0 μM and 1.0 mg/mL liver S9 protein in Tris buffer (pH 6.0). Samples at various times (0, 60, 120, 240, 360 and 1440 minutes) were removed, and immediately mixed with internal standard solution, phenol solution and dichloromethane solution. Samples were analyzed by LC-MS/MS; disappearance of test compound will be assessed based on peak area ratios of analyte/IS (no standard curve).

### Example 1: Design of YTHDF2 Inhibitor Decoy Oligonucleotides by Altering the Binding Site

YTHDF2 is a known epitranscriptome “reader” protein which binds to the RNA modification N6 methyladenosine (m6A) and targets bound RNAs for degradation. Inhibition of YTHDF2 should cause RNA stabilization. YTHDF2's canonical binding sequence, GGm6ACU, is known. CLIP-Seq data was used to identify other degenerate sequences recognized by YTHDF2. A previously disclosed YTHDF2 binding RNA oligo was used as a positive control (AAAAGGm6ACUGCUUUU, SEQ ID NO: 1; from Dominissini et al., 2012, “Topology of the human and mouse m6A RNA methylomes revealed by m6A-seq”, Nature, April 29; 485 (7397):201-6, herein incorporated by reference in its entirety). Table 1 summarizes the various degenerate binding sites tested.

In order to characterize the binding affinity of these decoy RNA oligos to YTHDF2 an ELISA-based cell free binding assay was developed. Briefly, wells of 96 well plates (NUNC MAXISORP, Thermo) were coated (overnight, 4° C.) with lug recombinant YTHDF2 YTH domain (amino acids 401-554 within SEQ ID NO: 63) in 100 μl of PBS and were then blocked with 5% BSA in PBS for 2 hours at room temperature. Samples in 100 μl sample buffer (PBS containing 0.1% BSA and 0.05% Tween 20) were loaded in serial dilution in duplicates and incubated for 1 hour at room temperature. HRP-Streptavidin (1:20,000) in sample buffer was added for 1 hour at room temperature and the reaction was visualized by the addition of 100 μl chromogenic substrate (TMB) for 15-20 min. The reaction was stopped with 100 μl H2SO4 (2 M) and absorbance at 450 nm was measured using an ELISA plate reader. Plates were washed four times with washing buffer (PBS containing 0.1% (v/v) Tween 20) after each step. As a reference for quantification, a standard curve was established by a serial dilution of the positive control RNA oligo (SEQ ID NO: 1). It should be noted that a molecule with the same sequence as SEQ ID NO: 1 (SEQ ID NO: 50) but lacking the m6A modification produced an estimated EC50 of 1048 nM, although it may have been even higher.

TABLE 1
Binding affinities of
different YTHDF2 binding sites

| SEQ | | EC50 |
|---|---|---|
| ID NO: | Sequence | (nM) |
| 1 | AAAAGGm6ACUGCUUUU | ~300 |
| 2 | AAAAGAm6ACUGCUUUU | 204.6 |
| 3 | AAAAGAm6ACCGCUUUU | 244.7 |
| 4 | AAAAGAm6ACAGCUUUU | 395.6 |
| 5 | AAAAAGm6ACUGCUUUU | 219.6 |
| 6 | AAAAAGm6ACCGCUUUU | 183 |
| 7 | AAAAAGm6ACAGCUUUU | 397.1 |
| 8 | AAAAGGm6ACCGCUUUU | 104.4 |
| 9 | AAAAGGm6ACAGCUUUU | 299.8 |

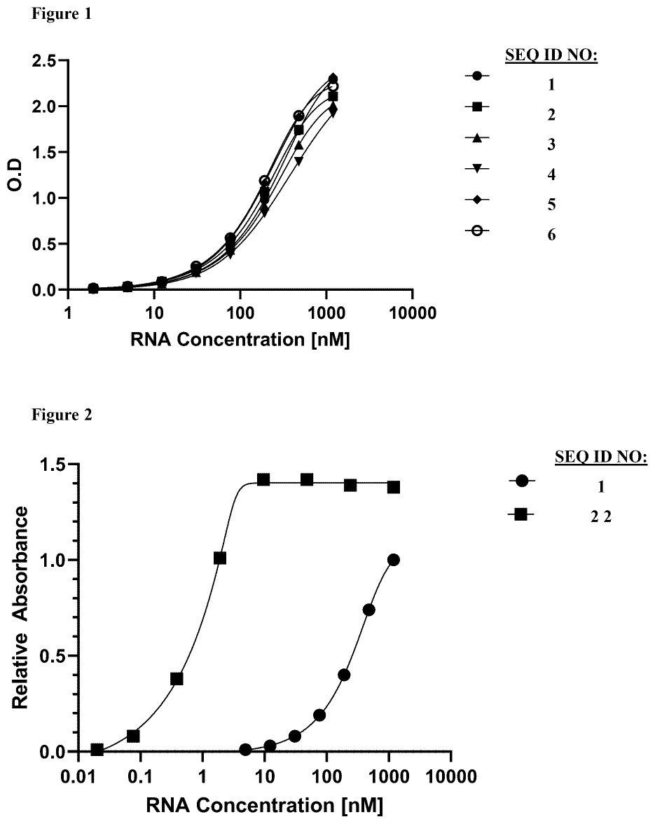

Eight alternative binding site sequences were tested, and while some produced a modest improvement in binding (SEQ ID NO: 2, 6 and 8) none of them even produced an order of magnitude improvement (FIG. 1). As such, repeating the canonical binding site more than once in the oligo was tested to see if this would improve binding efficiency. To this end a 20-mer RNA oligo was constructed that consisted of four repeats of the canonical sequence GGACU. No spacers were placed between the various repeats. In the control molecule (SEQ ID NO: 10) none of the adenine bases were m6 modified. Molecules with only one m6 modification, two modifications, three modifications or four modifications (all possible) were also tested. There is some low-level binding of YTHDF2 to the unmodified binding site, and so the use of four such binding sites produced binding that was essentially equivalent to the control molecule with a single modified binding site (SEQ ID NO: 1). Not surprisingly, it was found that including two copies of the binding site did indeed increase binding efficiency over a single copy. Three copies produced an even stronger result, and four copies produced the best effect (Table 2). Still the increase produced by using 4 repeats was not even a full order of magnitude. Interestingly, placing the binding sites in the middle of the molecule or closer to the periphery did not seem to have an appreciable effect on binding.

TABLE 2
Binding affinities of oligos with
increasing numbers of binding sites.

| SEQ | | No. | EC50 |
|---|---|---|---|
| ID NO: | Sequence | m6A | (nM) |
| 10 | GGACUGGACUGGACUGGACU | 0 | 269.9 |
| 11 | GGm6ACUGGACUGGACUGGACU | 1 | 94.81 |
| 12 | GGACUGGm6ACUGGACUGGACU | 1 | 82.06 |
| 13 | GGm6ACUGGm6ACUGGACUGGACU | 2 | 54.76 |
| 14 | GGACUGGm6ACUGGm6ACUGGACU | 2 | 22.96 |
| 15 | GGm6ACUGGm6ACUGGm6ACUGGACU | 3 | 20.98 |
| 16 | GGm6ACUGGm6ACUGGm6ACUGGm6ACU | 4 | 6.423 |

Next, rather than repeating the six-nucleotide binding motif, the sequences directly surrounding the binding motif in actual targets of YTHDF2 were incorporated into the RNA oligo and binding was evaluated. Based on the CLIP-Seq data four new RNA oligos were designed (SEQ ID NO: 17-20). In these molecules different binding motifs directly flanked by their endogenous flanking sequences observed in nature were used.

The results of the assay are provided in Table 3. The alternative direct flanking sequences did not significantly increase binding efficiency although the sequences used in SEQ ID NO: 17-19 showed minor improvements over the same binding sites flanked by AAAA and UUUU (see Table 1), though not as great an improvement was observed when including four binding site repeats.

TABLE 3
Binding affinities of RNA oligos
with modified direct flanking sequences.

| SEQ | | EC50 |
|---|---|---|
| ID NO: | Sequence | (nM) |
| 1 | AAAAGGm6ACUGCUUUU | 300 |
| 17 | GUCAAGUGGm6ACUUUG | 177.8 |
| 18 | ACAAGm6ACAGCCCUGU | 121.7 |
| 19 | CCUUGAGm6ACCUAGUA | 79.47 |
| 20 | CUCUUGGGAGm6ACAUA | 338.6 |

### Example 2: Design of YTHDF2 Inhibitor Decoy Oligonucleotides by Addition of Upstream/Downstream Motifs

Rather than looking at direct flanking sequence, the CLIP-Seq data was scanned for upstream and downstream motifs. Motifs enriched in the CLIP-Seq data were tested by generating RNA oligos comprising the motifs upstream and downstream relative to the YTHDF2 binding motif. The effect of spacer sequences before and after these new motifs was also examined. These spacers were randomly generated and positioned the flanking sequences such that they were still internal sequences and not at the very edge of the molecules. The results are summarized in Table 4. Of the seven 5′ flanking motifs tested (Sequences A-E and SEQ ID NO: 43-44) five of them produced at least one order of magnitude improvement in binding. The other two sequences (UCAGAGUC/Sequence B and SEQ ID NO: 43) did not produce such a strong improvement. Notably, the motif GGGAAACU (Sequence A) produced by far the best effect. Regardless of the 3′ motif used, the spacer used or even when no spacer and/or 3′ motif was used, when this motif was placed 5′ to the YTHDF2 binding site a two order of magnitude increase in binding efficiency was observed. This unexpected effect was by far the strongest effect observed.

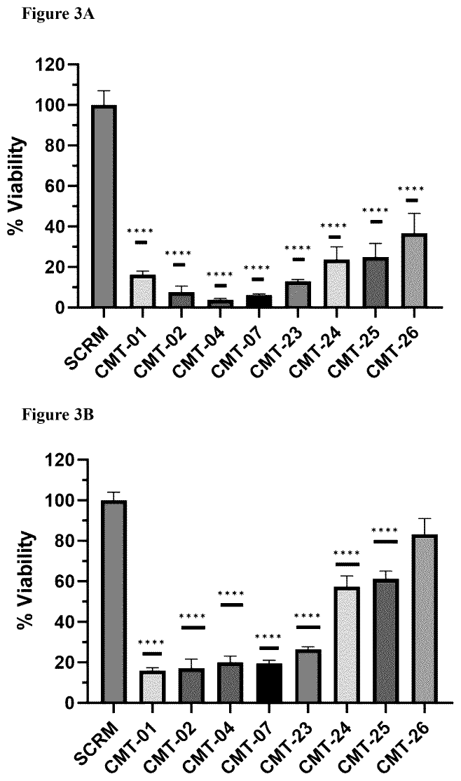

Five different 3′ flanking motifs were also tested (SEQ ID NO: 45-49). All of them were found effective in combination with the 5′ flanking motif GGGAAACU, although SEQ ID NO: 45, 46 and 49 were slightly superior (FIG. 2). However, these 3′ flanking motifs did not produce any improvement as compared to the control oligo when the constructs contained the ineffective 5′ motifs (SEQ ID NO: 26-27). Indeed, two oligos (SEQ ID NO: 34-35) completely lacking any 3′ sequence after the YTHDF2 binding site but containing the 5′ motif of GGGAAACU still produced a 2 order of magnitude increase over the control oligo. Thus, while the 3′ flanking motif may have a minor effect on binding efficiency, it is clear that the 5′ motif is far more important.

Next, it was checked if repeating the YTHDF2 binding site would enhance binding efficiency. Since GGGAAACU was so efficient in enhancing binding, it was included 5′ to both binding sites. Surprisingly, a repetition of the binding site had no effect on binding efficiency and the inclusion of the 3′ motifs produced improved results as compared to the repetition.

TABLE 4
Binding affinities of RNA oligos with
upstream and/or downstream flanking sequences.
Canonical binding site-bold;
5′ motif-underlined; 3′ motif-italicized;
spacer sequence-unmodified.

| SEQ | | 5′motif | 3′motif | |
|---|---|---|---|---|
| ID | | SEQ ID | SEQ ID | EC50 |
| NO: | Sequence | NO: | NO: | (nM) |
| 1 | AAAAGGm6ACUGCUUUU | — | — | 300 |
| 21 | UAGGGAAACUAACGGm6ACUCACAGACCUGGCUCG | A | 45 | 0.9734 |
| 22 | CUGGGAAACUGCGGGm6ACUGGGGAGCAUCUGCAC | A | 46 | 0.9205 |
| 23 | CUGGGAAACUGCGGGm6ACUUAGGCUGCUGCUGCUUA | A | 47 | 1.528 |
| 24 | CUGGGAAACUGCGGGm6ACUAACUGUGCAGCCCUCUG | A | 48 | 1.138 |
| 25 | CUGGGAAACUGCGGGm6ACUGAGCUGGGCCUGGGCAG | A | 49 | 0.3251 |
| 26 | CAUCAGAGUCAAUGGm6ACUCGAAGACCUGGCUCA | B | 45 | 124.6 |
| 27 | AAUCAGAGUCUUCGGm6ACUCGCGAGCAUCUGCAU | B | 46 | 367.2 |
| 28 | ACCUGCUGCUAUCGGm6ACUCACAGACCUGGCUCG | C | 45 | 31.33 |
| 29 | GCGGAGAGCUAAAGGm6ACUCACAGACCUGGCUCG | D | 45 | 18.62 |
| 30 | UAUGGCCACCAGCGGm6ACUCACAGACCUGGCUCG | E | 45 | 22.79 |
| 31 | CUAUGAGUCCAACAACGGm6ACUCACAGACCUGGCUCG | 43 | 45 | 235 |
| 32 | CUUGAACUUUGCGAACGGm6ACUCACAGACCUGGCUCG | 44 | 45 | 11.4 |
| 33 | GGGAAACUGGm6ACUGAGCAUCUGC | A | 46 | 1.731 |
| 34 | UAGGGAAACUAACGGm6ACU | A | — | 2.83 |
| 35 | CUGGGAAACUGCGGGm6ACU | A | — | 1.061 |
| 36 | CUGGGAAACUGCGGGm6ACUCUGGGAAACUGCGGGm6ACU | A | — | 0.9576 |
| 37 | GGGAAACUGGm6ACUGGGAAACUGGm6ACU | A | — | 2.457 |

### Example 3: Other Alterations in the Decoy Molecules

The sequence used to test binding site repeats is very G-rich due to ⅖ths of the bases in the binding site being guanine. It was therefore hypothesized that G-quadraplex secondary structure produced by G-G binding might have an effect on YTHDF2 binding. To test this, the 4-binding site molecules with single m6A modifications (SEQ ID NO: 11-12) were compared to molecules with the binding site in the same location, but with the non-binding site guanines replaced with uracils (SEQ ID NO: 51-52). The results are summarized in Table 5. The presence or absence of G-quadraplex structure did not have a substantial effect on binding.

TABLE 5
G-quadraplex effect on binding

| SEQ | | G- | EC50 |
|---|---|---|---|
| ID NO: | Sequence | quadraplex | (nM) |
| 11 | GGm6ACUGGACUGGACUGGACU | + | 94.81 |
| 12 | GGACUGGm6ACUGGACUGGACU | + | 82.06 |
| 51 | GGm6ACUUACGCAUUCCUCUAG | − | 51.37 |
| 52 | UUGACGGm6ACUAUUCCUCUAG | − | 171.8 |

Next the length of the spacer used was examined. Once again, the 4 binding site molecule in which each binding site includes a m6A modification was used (SEQ ID NO: 16). This molecule has no spacers between each binding site and still showed very efficient binding to YTHDF2. Spacers of length 2 bases, 4 bases and 6 bases were tested. As can be seen in Table 6, the spacer length did not have an effect on binding efficiency.

TABLE 6
Binding affinities of oligos
with different spacer lengths.

| SEQ | spacer | | EC50 |
|---|---|---|---|
| ID NO: | length | Sequence | (nM) |
| 16 | 0 | GGm6ACUGGm6ACUGGm6ACUGG | 6.423 |
| | | m6ACU | |
| 53 | 2 | GGm6ACUAUGGm6ACUAUGGm6A | 8.924 |
| | | CUAUGGm6ACU | |
| 54 | 4 | GGm6ACUGAUAGGm6ACUAGUAG | 1.816 |
| | | Gm6ACUUGACGGm6ACU | |
| 55 | 6 | GGm6ACUGAUAACGGm6ACUUCA | 5.313 |
| | | GUAGGm6ACUGUUGACGGm6ACU | |

Finally, the effect of different chemistries in the oligos backbone on binding efficiency were tested. A plain RNA oligo of SEQ ID NO: 1 was compared to oligos with phosphorothioate (PS) linkages in the backbone or 2-O-methylation in the backbone. These linkages are commonly added to RNA therapeutics as they confer nuclease resistance. Two PS modified RNA oligos were tested, one in which all the linkages were PS-linkages (SEQ ID NO: 56) and one in which the binding motif was left unmodified while the rest of the oligo was modified (SEQ ID NO: 57). Similarly, two 2-O-Me modified oligos were tested, one with the modification throughout the backbone (SEQ ID NO: 58) and one in which the binding site was left unmodified (SEQ ID NO: 59). As can be seen in Table 7, the 2-O-Me modification when present in the oligo in regions outside the binding site had no effect on YTHDF2 binding, but presence of the modification within the binding site completely abolished binding. In contrast, both PS modified oligos unexpectedly showed an increase in binding efficiency that was of two orders of magnitude. Quite surprisingly, the presence of the PS modification within the binding site did not abrogate binding as the 2-O-Me modification had.

A full DNA oligo was also tested (SEQ ID NO: 60), along with chimeric oligos in which the m6A was still an RNA base (SEQ ID NO: 61) or the whole binding site was still made from RNA bases (SEQ ID NO: 62). The full DNA molecule actually showed an order of magnitude increase in binding efficiency, while the chimeric molecules showed more moderate increases.

TABLE 7
Effects of different backbone chemistries on
binding affinity.

| SEQ | | | EC50 |
|---|---|---|---|
| ID NO: | Remarks | Sequence | (nM) |
| 1 | RNA | rArArArArGrGm6rA | ~300 |
| | | rCrUrGrCrUrUrUrU | |
| 56 | All PS | rA*rA*rA*rA*rG*rG* | 0.5102 |
| | | m6rA*rC*rU*rG*rC* | |
| | | rU*rU*rU*rU | |
| 57 | All PS | rA*rA*rA*rArGrG | 1.465 |
| | except | m6rArCrUrG*rC*rU* | |
| | binding | rU*rU*rU | |
| | motif | | |
| 58 | All 2′OMe | mAmAmAmAmGmGm6rA | — |
| | | mCmUmGmCmUmUmUmU | |
| 59 | All 2′OMe | mAmAmAmArGrGm6rA | 238 |
| | except | rCrUmGmCmUmUmUmU | |
| | binding | | |
| | motif | | |
| 60 | All DNA | AAAAGGm6ACTGCTTT | 18.32 |
| | | T | |
| 61 | All DNA | AAAAGGm6rACTGCTT | 82.27 |
| | but m6A | TT | |
| 62 | All DNA | AAAArGrGm6rArCrU | 100.5 |
| | but | GCTTTT | |
| | binding | | |
| | motif | | |

(r)-RNA base;
(*) PS linkage;
(m) 2-O-Me modified base;
all other bases are DNA bases.

### Example 4: Anticancer Effects of the Decoy Molecules

The ability of eight decoy molecules (Table 8) to inhibit tumor cell viability was tested in two triple-negative breast cancer cell lines: MDA-MD-468, and MDA-MB-231. Oligos CMT-01, CMT-02, CMT-04, CMT-07, and CMT-23 were designed based on the 5′ GGGAAACU motif, while oligos CMT-24, CMT-25, and CMT-26 were constructed using the 5′ motifs GGAGAGCU, UGGCCACC and AUGAGUCCAAC (SEQ ID NO: 44), respectively. The selected oligos, or similar oligos with these motifs, displayed EC50 values ranging from 0.3 to 31 nM in the cell-free binding assay (Table 4). In this initial analysis, the impact of the decoy oligos was investigated in vitro, and therefore no stabilizing backbone modifications were generated that could potentially influence binding. A scrambled oligo without a YTHDF2 bindins site was used as a negative control (CCCUUCUGUCUCCACCUCCC, SEQ ID NO: 64).

TABLE 8
RNA oligos used for anticancer analysis

| | SEQ ID | |
|---|---|---|
| Name | NO: | Sequence |
| CMT1 | 35 | CUGGGAAACUGCGGGm6ACU |
| CMT2 | 36 | CUGGGAAACUGCGGGm6ACU |
| | | CUGGGAAACUGCGGGm6ACU |
| CMT4 | 38 | GGm6ACUGGGAAACUGGm6A |
| | | CUGGGAAACUGGm6ACUGGG |
| | | AAACUGGm6ACU |
| CMT7 | 39 | GGGAAACUGGm6ACUGGGAA |
| | | ACUGGm6ACUGGGAAACUGG |
| | | m6ACU |
| CMT23 | 25 | CUGGGAAACUGCGGGm6ACU |
| | | GAGCUGGGCCUGGGCAG |
| CMT24 | 40 | GCGGAGAGCUAAAGGm6ACU |
| CMT25 | 41 | UAUGGCCACCAGCGGm6ACU |
| CMT26 | 42 | CUUGAACUUUGCGAACGGm6 |
| | | ACU |

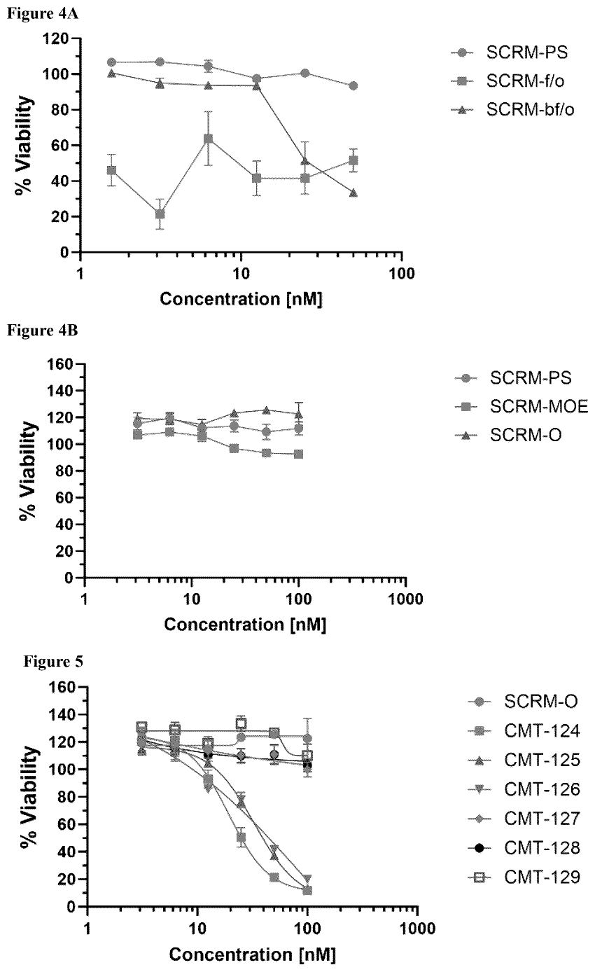

Due to the inherent instability of unmodified oligos, a triple transfection approach (200 nM) was employed over three consecutive days. Cell viability measurements were conducted using the CTG assay 48 hours after the last transfection. All eight tested oligos were able to significantly inhibit MDA-MB-468 cell viability (FIG. 3A), while all the oligos save CMT-26 significantly inhibited MDA-MB-231 cell viability (FIG. 3B). The five oligos based on the GGGAAACU motif were found to be superior to the other three tested oligos which mirrors the binding superiority observed for this motif. The difference was particularly pronounced in the MDA-MB-231 cells. CMT-01 and CMT-02 showed the greatest inhibition effect across both tested cell lines (84% and 82% decrease in viability in MDA-MB-231 and 83% and 92% decrease in MDA-MB-468). CMT-01 is a shorter sequence than CMT-02 (18 nucleotides vs. 36 nucleotides), and indeed was the shortest sequence tested A shorter length is advantage in terms of production efficiency and cost and so CMT-01 was selected as the focus of all future experiments.

### Example 5: Stabilizing Chemistry Optimization

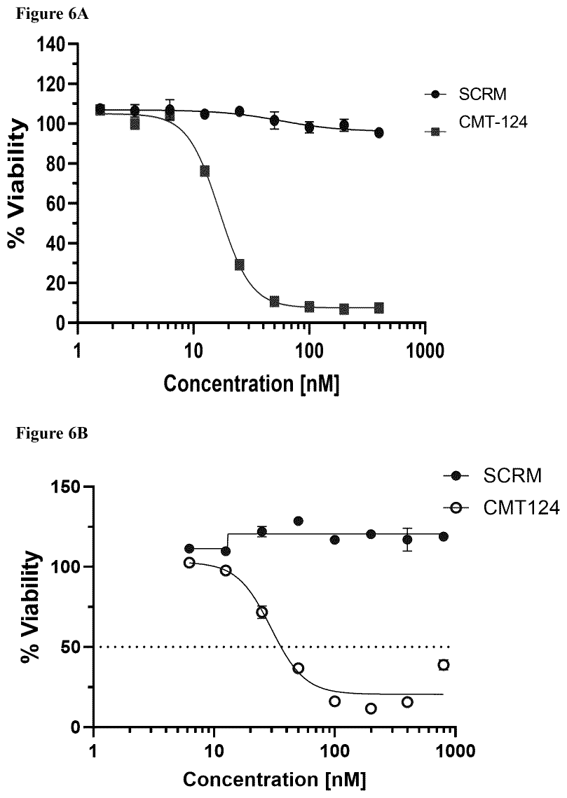

The ability of various stabilizing chemistries to improve CMT-01 were tested. At first oligonucleotides were designed with various established stabilizing chemistry modifications for RNA, including Phosphorothioate RNA bases (PS), 2′ Fluoro (2′F), 2′O Methyl(2′M), and DNA bases. Three control scrambled sequences were also generated, one with just PS modifications throughout, and two with PS modifications, 2′F modification and 2′O modifications (one had alternating modifications and one had blocks of modifications). Quite surprisingly it was found that incorporation of the 2′F modification in the decoy oligo resulted in cellular toxicity and reduced viability in MDA-MD-231 cells (FIG. 4A). This indicates that the combination of PS bases and 2′F bases is generally toxic and thus unsuitable for the decoy molecules as it would have a toxic effect on healthy cells as well.

As the PS modification was not found to be toxic, its combination with just 2′O methyl modified bases or just 2-methyoxyethyl modified bases was examined. None of the scrambled oligos with these modifications reduced viability in the MDA-MD-231 cells (FIG. 4B) and so oligos based on CMT-01 were designed with these modifications.

6 oligonucleotides (CMT-124-CMT-129, Table 9) were designed based on CMT-01. These oligonucleotides were constructed to be even shorter, having the 3-nucleotide spacer between the 5′ motif and the binding site removed. This resulted in the sequence CUGGGAAACUGGmACT (SEQ ID NO: 65). To safeguard the GGm6ACU binding site from exonuclease activity, three extra nucleotides (GAG) were appended to the 3′ end of the oligos resulting in an oligonucleotide of the sequence CUGGGAAACUGGmACUGAG (SEQ ID NO: 66). These protective measures were tested due to the possible susceptibility of the ends to exonucleolytic degradation.

The six oligos all contained phosphorothioate linkages throughout. In addition, certain decoy oligos were strategically engineered to feature DNA bases at the GGACU binding motif of YTHDF2 (CMT-124, 125, 127, 129), facilitating an assessment of their impact on stabilizing the binding to YTHDF2. Three of the oligos contained 2′O methyl modified bases (CMT-124-126) and three contained 2-MOE modified bases (CMT-127-129).

TABLE 9
CMT-01 with various stabilizing
chemistries and 3 base 3′ extension.

| | SEQ | | |
|---|---|---|---|
| ID | ID | Modified Nucleotides | EC50 |
| CMT- | 67 | 2mC*2mU*2mG*2mG*2mG* | 19.32 |
| 124 | | 2mA*2mA*2mA*2mC*2mU* | |
| | | G*G*m6A*C*T*2mG*2mA* | |
| | | 2mG | |
| CMT- | 68 | 2mC*2mU*2mG*G*G*A*A* | 33.04 |
| 125 | | 2mA*2mC*2mU*G*G*m6A* | |
| | | C*T*2mG*2mA*2mG | |
| CMT- | 69 | 2mC*2mU*2mG*G*G*A*A* | 105.7 |
| 126 | | 2mA*2mC*2mU*2mG*2mG* | |
| | | m6A*2mC*2mU*2mG*2mA* | |
| | | 2mG | |
| CMT- | 70 | MOErC*MOErU*MOErG* | — |
| 127 | | MOErG*MOErG*MOErA* | |
| | | MOErA*MOErA*MOErC* | |
| | | MOErU*G*G*m6A*C*T* | |
| | | MOErG*MOErA*MOErG | |
| CMT- | 71 | MOErC*MOErU*MOErG* | — |
| 128 | | MOErG*MOErG*MOErA* | |
| | | MOErA*MOErA*MOErC* | |
| | | MOErU*MOErG*MOErG* | |
| | | m6A*MOErC*MOErU* | |
| | | MOErG*MOErA*MOErG | |
| CMT- | 72 | MOErC*MOErU*MOErG* | — |
| 129 | | G*G*A*A*MOErA* | |
| | | MOErC*MOErU*G*G* | |
| | | m6A*C*T*MOErG* | |
| | | MOErA*MOErG | |

m = m6,
* = Phosphorothioated RNA bases,
2m = 2′Ome,
MOEr = 2-Methoxyethyl,
bold = DNA bases

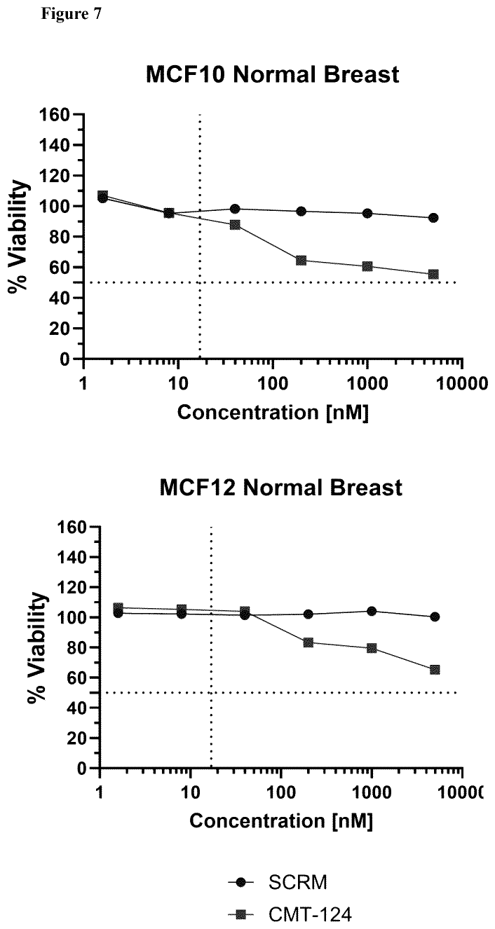

To test the efficacy of these oligos, MDA-MB-231 cells were transfected with the oligos and a dose-response analysis for cell viability was conducted for each molecule (FIG. 5). Oligos CMT-127-129, incorporating the bulky 2′MOE modification, did not produce an effect on cell viability. This observation is likely attributable to the bulkiness of the modification, which potentially disrupts oligo-YTHDF2 protein interactions. Conversely, oligos CMT 124-126, featuring the less bulky 2′M modification, exhibited a notable dose-dependent effect. EC50 values were determined based on the viability reduction (Table 9). This analysis indicated that oligos CMT-124-125, where the binding site GGm6ACU comprised DNA nucleotides, elicited a stronger effect (lower EC50) than CMT-126, where the binding site GGm6ACU was adorned with the 2′methyl modification (which is a bulkier modification than the 2′H in DNA). CMT-124 was notably the best oligo and so it was selected for further testing.

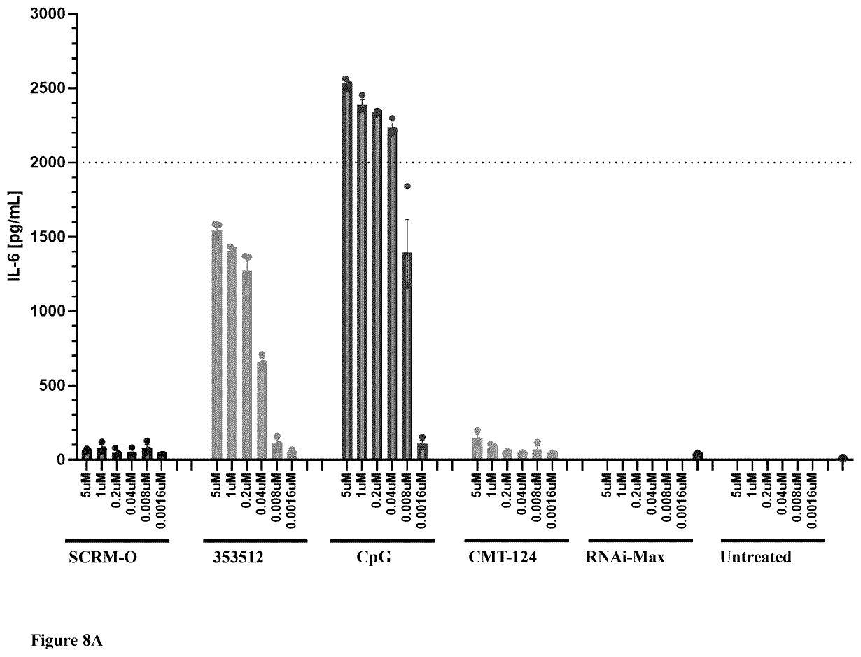

CMT-124 was tested in MDA-MD-231 cells at higher concentrations, though its effect reached its maximum already at 100 nM (FIG. 6A). It was found to be highly effective in a patient-derived triple negative breast cancer cell line (MAXFTN) as well (FIG. 6B). The ability of CMT-124 to reduce viability was also tested in other triple negative cell lines (MDA-MB-468, HCC1937 and HS578T), lung cancer cell lines (A549, H1299, H1650), a lung patient derived cell line (LXFN), liver cancer cell line (Hep-3B, Huh7D), a pancreatic cancer cell line (MIA-PaCa-2), a tongue cancer cell line (Cal27), brain cancer cell lines (U87, DAOY, SK-N-BE (2), IMR-32), uterine cancer cell lines (SiHa, HeLa), a muscle cancer cell lines (RMS13), and kidney cancer cell lines (769-P, 786-0). This decoy oligo was found to be effective in all cell lines tested and at similar levels (Table 11).

TABLE 11
CMT-124 effect on viability in various cancers

| Cell line | Tissue | Disease | EC50 [nM] |
|---|---|---|---|
| MDA-MB-231 | Breast | Adenocarcinoma | 19.3 |
| MDA-MB-468 | Breast | Adenocarcinoma | 18.81 |
| HCC1937 | Breast | Ductal | 17.12 |
| | | carcinoma | |
| A549 | Lung | carcinoma | 34.58 |
| H1299 | Lung | carcinoma | 46.65 |
| H1650 | Lung | Bronchoalveolar | 21.62 |
| | | carcinoma | |
| Hep3b | liver | Hepatocellular | 26.82 |
| | | Carcinoma | |
| MAXFN (PDX) | Breast | Mammary | 29.73 |
| LXFN(PDX) | Lung | NSCLC | 40.09 |
| MIA-PaCa-2 | Pancreas | Carcinoma | 52.51 |
| Cal27 | Tongue | Squamous | 44.53 |
| | | Cell Carcinoma | |
| U87 | Brain | Glioblastoma | 17.13 |
| DAOY | Brain; | Desmoplastic | 46.23 |
| | Cerebellum | Cerebellar | |
| | | Medulloblastoma | |
| SiHa | Uterus; | Squamous Cell | 34 |
| | Cervix | Carcinoma | |
| Hela | Uterus; | Adenocarcinoma | 32.13 |
| | Cervix | | |
| RMS13 | Muscle | Rhabdomyosarcoma | 45.17 |
| 769-P | Kidney | Adenocarcinoma; | 47.73 |
| | | Renal Cell | |
| 786-O | Kidney | Adenocarcinoma; | 56.19 |
| | | Renal Cell | |
| SK-N-BE(2) | Brain | Neuroblastoma | 65.665 |
| Huh7D | Liver | Hepatocellular | 38.53 |
| | | Carcinoma | |
| IMR-32 | Brain | Neuroblastoma | 55.73 |

### Example 6: Decoy Oligos have No Effect on Healthy Cells

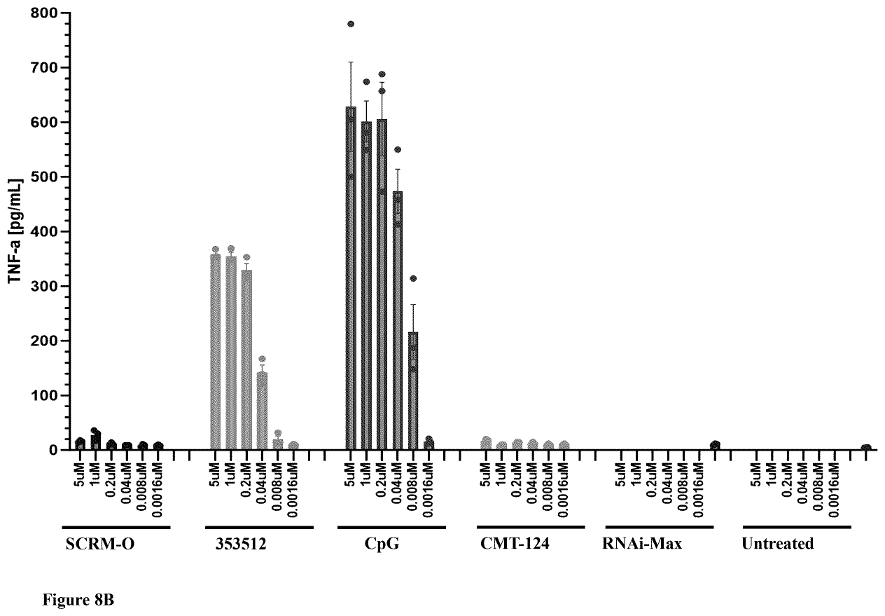

CMT-124 was not expected to have a substantial effect on healthy control cells. To test this, viability was measured in MCF10 and MCF12 normal breast cells cultured in the presence of increasing concentrations of CMT-124. As can be seen in FIG. 7, even at the maximum concentration tested greater than 50% viability was retained in the healthy cells. Further, at a concentration of 100 nm which was sufficient to elicit maximum killing in cancer cells, 80% viability was retained in healthy cells.

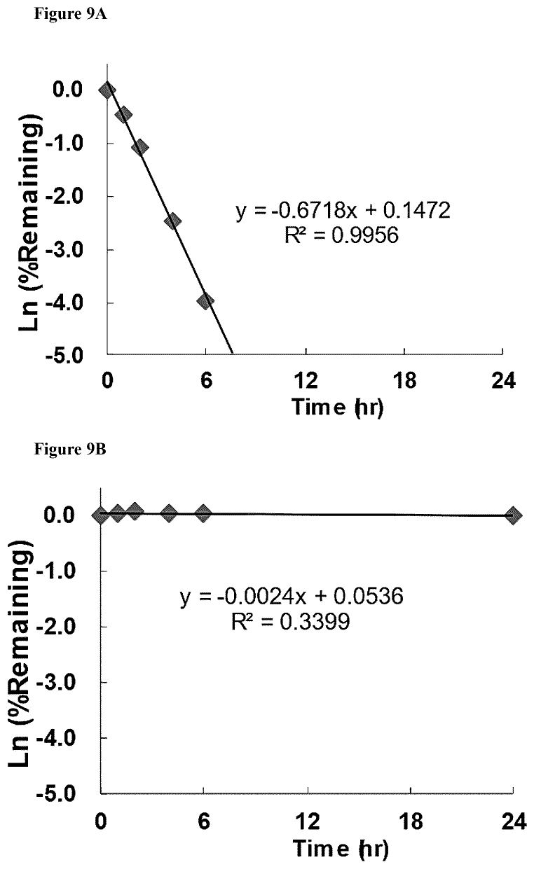

As CMT-124 is an artificial sequence there was a concern that it might induce a host immune response. To test this human PBMCs were cultured with the decoy oligo and secretion of proinflammatory cytokines IL-6 and TNF-α was measured. PBMCs were treated with a control oligonucleotide known to induce immune response (oligo 353512) or a CpG repeat oligo. Scrambled sequence was used as a negative control, along with transfection reagent only and a no treatment control. CMT-124 did not induce an innate immune response from the PBMCs with both IL-6 (FIG. 8A) and TNF-α (FIG. 8B) levels remaining at baseline.

### Example 7: Decoy Oligos are Highly Stable in Human Plasma and Liver

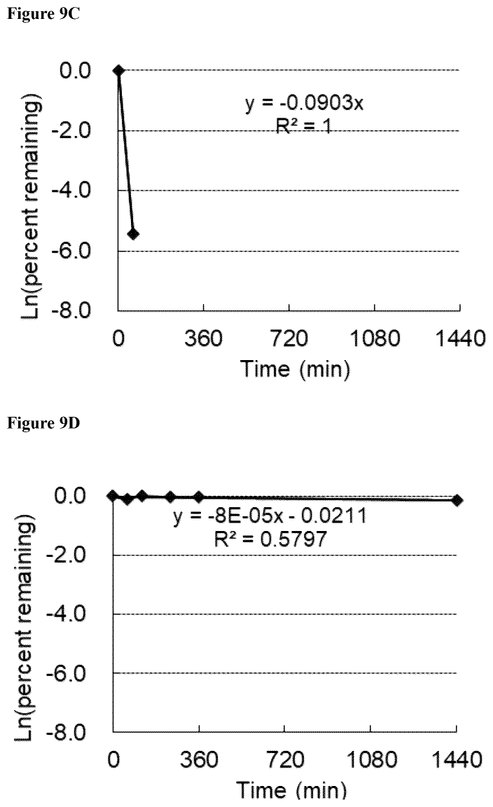

The phosphorothioate linkage and 2′O methyl modification are both known to increase RNA stability and slow degradation when administered to subjects. To test this, CMT-124 was incubated in human plasma for 24 hours and a sample was extracted at 0, 1, 2, 4, 6 and 24 hours. The sample was assessed by LC-MS/MS to measure disappearance of the test compound. The internal positive control (CCGGAATTCATGCTCAGACCTATCT, SEQ ID NO: 73) was observed to disappear over time in the plasma (FIG. 9A), by contrast CMT-124 showed very high stability with 98.9% remaining in plasma after 24 hr. (FIG. 9B).

A similar assay was performed in S9 liver extract. Significant drug metabolism occurs in the liver and the S9 extract contains active liver enzymes and is an effective model for liver metabolism. Degradation was measured in the liver extract over a total of 1440 minutes. As before, the internal control showed active degradation (FIG. 9C). In contrast, CMT-124 was high stability with 86.5 remaining in S9 liver extract after 24 hr. (FIG. 9D). This indicates that the chemical modifications to the RNA are effective in protecting it from degradation, both in the blood and in the liver.

The efficacy of the various decoy molecules is tested in further cancer cell lines that are known to feature mRNA instability and healthy counterpart tissue that does not. Lines that overexpress and do not overexpress YTHDF2 are tested. Some of the following cell lines are tested: AML lines: THP-1, Kasumi, and MV-4-11; lung cancer lines: A549, H1650, H596, H1437, H1299, and H366; and breast cancer lines: MD-MBA-231, MD-MBA-468, and MD-MBA-453. Addition of molecules with an irrelevant sequence, the binding site with an m6A modification, without flanking motifs or without backbone modification are used as negative controls. The molecule of Dominissini is also used as a benchmark for comparison. The level of mRNA stability in the cells is measured as is cell survival/proliferation. The decoy molecules are found to increase mRNA half-life of YTHDF2 targets and to increase cancer cell death/reduce proliferation in many of the cell lines in a dose dependent manner.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A nucleic acid molecule comprising a YTH domain-containing family protein 2 (YTHDF2) binding site and a 5′ enhancer motif located 5′ to said binding site, wherein said 5′ enhancer motif is selected from GGGAAACU, CUGCUGCU, GGAGAGCU, UGGCCACC and AUGAGUCCAAC (SEQ ID NO: 44) and wherein said molecule comprises 13-100 nucleotides.

2. A nucleic acid molecule comprising a YTHDF2 binding site, wherein said molecule comprises 10-100 nucleotides and a phosphorothioate (PS) modified backbone.

3. The nucleic acid molecule of claim 2, comprising a 5′ enhancer motif located 5′ to said binding site, wherein said 5′ enhancer motif is selected from GGGAAACU, CUGCUGCU, GGAGAGCU, UGGCCACC and SEQ ID NO: 44.

4. (canceled)

5. The nucleic acid molecule of claim 1, wherein said binding site consists of a sequence selected from GGACU, GAACU, GAACC, GAACA, AGACU, AGACC, AGACA, GGACC, GGACA, AAACU, AAACA, AAACG, UAACU, UAACA, UAACG, UGACU, UGACA and UGACG and wherein the central adenosine in said binding site is a N6 methyladenosine, optionally wherein said binding site consists of GGACU and said adenosine is a N6 methyladenosine.

6. (canceled)

7. The nucleic acid molecule of claim 1, wherein said 5′ enhancer motif and said binding site are separated by a first spacer, optionally said first spacer comprises 1-6 nucleotides.

8. (canceled)

9. The nucleic acid molecule of claim 1, wherein said molecule is a single stranded RNA molecule, a single stranded DNA molecule, or a chimeric molecule comprising both DNA and RNA bases, optionally wherein said binding site is DNA bases and all non-binding site bases are RNA bases.

10. (canceled)

11. (canceled)

12. (canceled)

13. (canceled)

14. The nucleic acid molecule of claim 1, comprising a chemically modified backbone comprising a phosphorothioate (PS) linkage.

15. (canceled)

16. The nucleic acid molecule of claim 14, comprising at least one of:
a. a fully PS-modified backbone;
b. a PS-modified backbone in which said binding site is not PS modified;
c. at least one 2′O methyl modified base; and
d. all RNA bases being 2′O methyl modified.

17. (canceled)

18. (canceled)

19. (canceled)

20. The nucleic acid molecule of claim 1, comprising the nucleotide sequence CUGGGAAACUGGmACUGAG (SEQ ID NO: 66), wherein mA is a N6 methyladenosine, comprising a nucleotide sequence consisting of SEQ ID NO: 66 or consisting of SEQ ID NO: 67.

21. (canceled)

22. (canceled)

23. The nucleic acid molecule of claim 1, further comprising a 3′ enhancer motif, wherein said 3′ enhancer motif is selected from SEQ ID NO: 45-49, optionally wherein said 3′ enhancer motif and said binding site are separated by a second spacer and optionally wherein said second spacer comprises 1-6 nucleotide.

24. (canceled)

25. (canceled)

26. The nucleic acid molecule of claim 1, further comprising a 5′ terminal spacer, a 3′ terminal spacer or both.

27. The nucleic acid molecule of claim 1, comprising 2, 3 or 4 binding sites.

28. The nucleic acid molecule of claim 27, wherein said binding sites comprise the same or different sequences, wherein said 2, 3 or 4 binding sites are separated by third spacers, wherein said 2, 3 or 4 binding sites are separated by third spacers comprising 1-6 nucleotides, or a combination thereof.

29. (canceled)

30. (canceled)

31. The nucleic acid molecule of claim 1, comprising or consisting of a sequence selected from SEQ ID NO: 21-25, 28-30, 32-42 and 65-66.

32. (canceled)

33. (canceled)

34. (canceled)

35. The nucleic acid molecule of claim 1, comprising or consisting of a sequence selected from SEQ ID NO: 1-9, 11-42, 51-55 and 65-66.

36. (canceled)

37. (canceled)

38. (canceled)

39. A pharmaceutical composition comprising a nucleic acid molecule of claim 1 and a pharmaceutically acceptable carrier excipient or adjuvant, optionally being formulated for administration to a subject.

40. (canceled)

41. (canceled)

42. (canceled)

43. (canceled)

44. (canceled)

45. (canceled)

46. (canceled)

47. A method of inhibiting YTHDF2, the method comprising contacting said YTHDF2 with a nucleic acid molecule of claim 1, thereby inhibiting YTHDF2, optionally wherein said inhibiting is inhibiting YTHDF2 mediated degradation of a m6A containing mRNA.

48. (canceled)

49. A method of treating a disease or condition characterized by YTHDF2 mediated mRNA instability or degradation in a subject in need thereof, the method comprising administering to said subject a pharmaceutical composition of claim 39, thereby treating a disease or condition, optionally wherein YTHDF2 mediated mRNA instability or degradation causes, sustains or enhances said disease or condition.

50. (canceled)

51. The method of claim 49, wherein said disease or condition is selected from cancer, or viral infection, optionally wherein said cancer is selected from lung cancer, liver cancer, brain cancer, renal cancer, muscle cancer, uterine cancer, cervical cancer, head and neck cancer, pancreatic cancer and breast cancer, or said viral infection is selected from a Kaposi's Sarcoma-associated Herpesvirus (KSHV), Human Cytomegalovirus (HCMV), Human Immunodeficiency Virus 1 (HIV-1) and influenza.

52. (canceled)

53. (canceled)

54. (canceled)

55. A method of determining suitability of a subject in need thereof, the method comprising, receiving a sample containing disease cells from the subject, and measuring YTHDF2 levels or mRNA instability or degradation in said sample, wherein the presence of YTHDF2 levels or mRNA instability or degradation above a predetermined threshold indicates the subject is suitable to be treated by a method of claim 49 and further comprising administering a pharmaceutical composition of claim 39 to a subject determined to be suitable, thereby determining suitability.

56. (canceled)

57. (canceled)


SEQUENCE LISTING (74 sequences)

SEQ ID NO: 1 - RNA, 15 nt, synthetic construct
  misc_feature 1..15: note = Synthetic
  source 1..15: mol_type = other RNA
  modified_base 7: mod_base = m6a
aaaaggactgctttt

SEQ ID NO: 2 - RNA, 15 nt, synthetic construct
  misc_feature 1..15: note = Synthetic
  source 1..15: mol_type = other RNA
  modified_base 7: mod_base = m6a
aaaagaactgctttt

SEQ ID NO: 3 - RNA, 15 nt, synthetic construct
  misc_feature 1..15: note = Synthetic
  source 1..15: mol_type = other RNA
  modified_base 7: mod_base = m6a
aaaagaaccgctttt

SEQ ID NO: 4 - RNA, 15 nt, synthetic construct
  misc_feature 1..15: note = Synthetic
  source 1..15: mol_type = other RNA
  modified_base 7: mod_base = m6a
aaaagaacagctttt

SEQ ID NO: 5 - RNA, 15 nt, synthetic construct
  misc_feature 1..15: note = Synthetic
  source 1..15: mol_type = other RNA
  modified_base 7: mod_base = m6a
aaaaagactgctttt

SEQ ID NO: 6 - RNA, 15 nt, synthetic construct
  misc_feature 1..15: note = Synthetic
  source 1..15: mol_type = other RNA
  modified_base 7: mod_base = m6a
aaaaagaccgctttt

SEQ ID NO: 7 - RNA, 15 nt, synthetic construct
  misc_feature 1..15: note = Synthetic
  source 1..15: mol_type = other RNA
  modified_base 7: mod_base = m6a
aaaaagacagctttt

SEQ ID NO: 8 - RNA, 15 nt, synthetic construct
  misc_feature 1..15: note = Synthetic
  source 1..15: mol_type = other RNA
  modified_base 7: mod_base = m6a
aaaaggaccgctttt

SEQ ID NO: 9 - RNA, 15 nt, synthetic construct
  misc_feature 1..15: note = Synthetic
  source 1..15: mol_type = other RNA
  modified_base 7: mod_base = m6a
aaaaggacagctttt

SEQ ID NO: 10 - RNA, 20 nt, synthetic construct
  misc_feature 1..20: note = Synthetic
  source 1..20: mol_type = other RNA
ggactggactggactggact

SEQ ID NO: 11 - RNA, 20 nt, synthetic construct
  misc_feature 1..20: note = Synthetic
  source 1..20: mol_type = other RNA
  modified_base 3: mod_base = m6a
ggactggactggactggact

SEQ ID NO: 12 - RNA, 20 nt, synthetic construct
  misc_feature 1..20: note = Synthetic
  source 1..20: mol_type = other RNA
  modified_base 8: mod_base = m6a
ggactggactggactggact

SEQ ID NO: 13 - RNA, 20 nt, synthetic construct
  misc_feature 1..20: note = Synthetic
  source 1..20: mol_type = other RNA
  modified_base 3: mod_base = m6a
  modified_base 8: mod_base = m6a
ggactggactggactggact

SEQ ID NO: 14 - RNA, 20 nt, synthetic construct
  misc_feature 1..20: note = Synthetic
  source 1..20: mol_type = other RNA
  modified_base 8: mod_base = m6a
  modified_base 13: mod_base = m6a
ggactggactggactggact

SEQ ID NO: 15 - RNA, 20 nt, synthetic construct
  misc_feature 1..20: note = Synthetic
  source 1..20: mol_type = other RNA
  modified_base 3: mod_base = m6a
  modified_base 8: mod_base = m6a
  modified_base 13: mod_base = m6a
ggactggactggactggact

SEQ ID NO: 16 - RNA, 20 nt, synthetic construct
  misc_feature 1..20: note = Synthetic
  source 1..20: mol_type = other RNA
  modified_base 3: mod_base = m6a
  modified_base 8: mod_base = m6a
  modified_base 13: mod_base = m6a
  modified_base 18: mod_base = m6a
ggactggactggactggact

SEQ ID NO: 17 - RNA, 15 nt, synthetic construct
  misc_feature 1..15: note = Synthetic
  source 1..15: mol_type = other RNA
  modified_base 10: mod_base = m6a
gtcaagtggactttg

SEQ ID NO: 18 - RNA, 15 nt, synthetic construct
  misc_feature 1..15: note = Synthetic
  source 1..15: mol_type = other RNA
  modified_base 6: mod_base = m6a
acaagacagccctgt

SEQ ID NO: 19 - RNA, 15 nt, synthetic construct
  misc_feature 1..15: note = Synthetic
  source 1..15: mol_type = other RNA
  modified_base 8: mod_base = m6a
ccttgagacctagta

SEQ ID NO: 20 - RNA, 15 nt, synthetic construct
  misc_feature 1..15: note = Synthetic
  source 1..15: mol_type = other RNA
  modified_base 11: mod_base = m6a
ctcttgggagacata

SEQ ID NO: 21 - RNA, 33 nt, synthetic construct
  misc_feature 1..33: note = Synthetic
  source 1..33: mol_type = other RNA
  modified_base 16: mod_base = m6a
tagggaaactaacggactcacagacctggctcg

SEQ ID NO: 22 - RNA, 33 nt, synthetic construct
  misc_feature 1..33: note = Synthetic
  source 1..33: mol_type = other RNA
  modified_base 16: mod_base = m6a
ctgggaaactgcgggactggggagcatctgcac

SEQ ID NO: 23 - RNA, 35 nt, synthetic construct
  misc_feature 1..35: note = Synthetic
  source 1..35: mol_type = other RNA
  modified_base 16: mod_base = m6a
ctgggaaactgcgggacttaggctgctgctgctta

SEQ ID NO: 24 - RNA, 35 nt, synthetic construct
  misc_feature 1..35: note = Synthetic
  source 1..35: mol_type = other RNA
  modified_base 16: mod_base = m6a
ctgggaaactgcgggactaactgtgcagccctctg

SEQ ID NO: 25 - RNA, 35 nt, synthetic construct
  misc_feature 1..35: note = Synthetic
  source 1..35: mol_type = other RNA
  modified_base 16: mod_base = m6a
ctgggaaactgcgggactgagctgggcctgggcag

SEQ ID NO: 26 - RNA, 33 nt, synthetic construct
  misc_feature 1..33: note = Synthetic
  source 1..33: mol_type = other RNA
  modified_base 16: mod_base = m6a
catcagagtcaatggactcgaagacctggctca

SEQ ID NO: 27 - RNA, 33 nt, synthetic construct
  misc_feature 1..33: note = Synthetic
  source 1..33: mol_type = other RNA
  modified_base 16: mod_base = m6a
aatcagagtcttcggactcgcgagcatctgcat

SEQ ID NO: 28 - RNA, 33 nt, synthetic construct
  misc_feature 1..33: note = Synthetic
  source 1..33: mol_type = other RNA
  modified_base 16: mod_base = m6a
acctgctgctatcggactcacagacctggctcg

SEQ ID NO: 29 - RNA, 33 nt, synthetic construct
  misc_feature 1..33: note = Synthetic
  source 1..33: mol_type = other RNA
  modified_base 16: mod_base = m6a
gcggagagctaaaggactcacagacctggctcg

SEQ ID NO: 30 - RNA, 33 nt, synthetic construct
  misc_feature 1..33: note = Synthetic
  source 1..33: mol_type = other RNA
  modified_base 16: mod_base = m6a
tatggccaccagcggactcacagacctggctcg

SEQ ID NO: 31 - RNA, 36 nt, synthetic construct
  misc_feature 1..36: note = Synthetic
  source 1..36: mol_type = other RNA
  modified_base 19: mod_base = m6a
ctatgagtccaacaacggactcacagacctggctcg

SEQ ID NO: 32 - RNA, 36 nt, synthetic construct
  misc_feature 1..36: note = Synthetic
  source 1..36: mol_type = other RNA
  modified_base 19: mod_base = m6a
cttgaactttgcgaacggactcacagacctggctcg

SEQ ID NO: 33 - RNA, 23 nt, synthetic construct
  misc_feature 1..23: note = Synthetic
  source 1..23: mol_type = other RNA
  modified_base 11: mod_base = m6a
gggaaactggactgagcatctgc

SEQ ID NO: 34 - RNA, 18 nt, synthetic construct
  misc_feature 1..18: note = Synthetic
  source 1..18: mol_type = other RNA
  modified_base 16: mod_base = m6a
tagggaaactaacggact

SEQ ID NO: 35 - RNA, 18 nt, synthetic construct
  misc_feature 1..18: note = Synthetic
  source 1..18: mol_type = other RNA
  modified_base 16: mod_base = m6a
ctgggaaactgcgggact

SEQ ID NO: 36 - RNA, 36 nt, synthetic construct
  misc_feature 1..36: note = Synthetic
  source 1..36: mol_type = other RNA
  modified_base 16: mod_base = m6a
  modified_base 34: mod_base = m6a
ctgggaaactgcgggactctgggaaactgcgggact

SEQ ID NO: 37 - RNA, 26 nt, synthetic construct
  misc_feature 1..26: note = Synthetic
  source 1..26: mol_type = other RNA
  modified_base 11: mod_base = m6a
  modified_base 24: mod_base = m6a
gggaaactggactgggaaactggact

SEQ ID NO: 38 - RNA, 44 nt, synthetic construct
  source 1..44: mol_type = other RNA
  modified_base 3: mod_base = m6a
  modified_base 16: mod_base = m6a
  modified_base 29: mod_base = m6a
  modified_base 42: mod_base = m6a
ggactgggaaactggactgggaaactggactgggaaactggact

SEQ ID NO: 39 - RNA, 39 nt, synthetic construct
  source 1..39: mol_type = other RNA
  modified_base 11: mod_base = m6a
  modified_base 24: mod_base = m6a
  modified_base 37: mod_base = m6a
gggaaactggactgggaaactggactgggaaactggact

SEQ ID NO: 40 - RNA, 18 nt, synthetic construct
  source 1..18: mol_type = other RNA
  modified_base 16: mod_base = m6a
gcggagagctaaaggact

SEQ ID NO: 41 - RNA, 18 nt, synthetic construct
  source 1..18: mol_type = other RNA
  modified_base 16: mod_base = m6a
tatggccaccagcggact

SEQ ID NO: 42 - RNA, 21 nt, synthetic construct
  source 1..21: mol_type = other RNA
  modified_base 19: mod_base = m6a
cttgaactttgcgaacggact

SEQ ID NO: 43 - RNA, 11 nt, synthetic construct
  misc_feature 1..11: note = Synthetic
  source 1..11: mol_type = other RNA
atgagtccaac

SEQ ID NO: 44 - RNA, 11 nt, synthetic construct
  misc_feature 1..11: note = Synthetic
  source 1..11: mol_type = other RNA
tgaactttgcg

SEQ ID NO: 45 - RNA, 10 nt, synthetic construct
  misc_feature 1..10: note = Synthetic
  source 1..10: mol_type = other RNA
agacctggct

SEQ ID NO: 46 - RNA, 10 nt, synthetic construct
  misc_feature 1..10: note = Synthetic
  source 1..10: mol_type = other RNA
gagcatctgc

SEQ ID NO: 47 - RNA, 12 nt, synthetic construct
  misc_feature 1..12: note = Synthetic
  source 1..12: mol_type = other RNA
gctgctgctgct

SEQ ID NO: 48 - RNA, 12 nt, synthetic construct
  misc_feature 1..12: note = Synthetic
  source 1..12: mol_type = other RNA
tgtgcagccctc

SEQ ID NO: 49 - RNA, 12 nt, synthetic construct
  misc_feature 1..12: note = Synthetic
  source 1..12: mol_type = other RNA
ctgggcctgggc

SEQ ID NO: 50 - RNA, 15 nt, synthetic construct
  misc_feature 1..15: note = Synthetic
  source 1..15: mol_type = other RNA
aaaaggactgctttt

SEQ ID NO: 51 - RNA, 20 nt, synthetic construct
  misc_feature 1..20: note = Synthetic
  source 1..20: mol_type = other RNA
  modified_base 3: mod_base = m6a
ggacttacgcattcctctag

SEQ ID NO: 52 - RNA, 20 nt, synthetic construct
  misc_feature 1..20: note = Synthetic
  source 1..20: mol_type = other RNA
  modified_base 8: mod_base = m6a
ttgacggactattcctctag

SEQ ID NO: 53 - RNA, 26 nt, synthetic construct
  misc_feature 1..26: note = Synthetic
  source 1..26: mol_type = other RNA
  modified_base 3: mod_base = m6a
  modified_base 10: mod_base = m6a
  modified_base 17: mod_base = m6a
  modified_base 24: mod_base = m6a
ggactatggactatggactatggact

SEQ ID NO: 54 - RNA, 32 nt, synthetic construct
  misc_feature 1..32: note = Synthetic
  source 1..32: mol_type = other RNA
  modified_base 3: mod_base = m6a
  modified_base 12: mod_base = m6a
  modified_base 21: mod_base = m6a
  modified_base 30: mod_base = m6a
ggactgataggactagtaggacttgacggact

SEQ ID NO: 55 - RNA, 38 nt, synthetic construct
  misc_feature 1..38: note = Synthetic
  source 1..38: mol_type = other RNA
  modified_base 3: mod_base = m6a
  modified_base 14: mod_base = m6a
  modified_base 25: mod_base = m6a
  modified_base 36: mod_base = m6a
ggactgataacggacttcagtaggactgttgacggact

SEQ ID NO: 56 - RNA, 15 nt, synthetic construct
  misc_feature 1..15: note = Synthetic
  source 1..15: mol_type = other RNA
  modified_base 1..15: mod_base = OTHER; note = phosphorothioate linkage
  modified_base 7: mod_base = m6a
aaaaggactgctttt

SEQ ID NO: 57 - RNA, 15 nt, synthetic construct
  misc_feature 1..15: note = Synthetic
  source 1..15: mol_type = other RNA
  modified_base 1..4: mod_base = OTHER; note = phosphorothioate linkage
  modified_base 7: mod_base = m6a
  modified_base 10..15: mod_base = OTHER; note = phosphorothioate linkage
aaaaggactgctttt

SEQ ID NO: 58 - RNA, 15 nt, synthetic construct
  misc_feature 1..15: note = Synthetic
  source 1..15: mol_type = other RNA
  modified_base 7: mod_base = m6a
  modified_base 1..15: mod_base = OTHER; note = 2' OMe linkage
aaaaggactgctttt

SEQ ID NO: 59 - RNA, 15 nt, synthetic construct
  misc_feature 1..15: note = Synthetic
  source 1..15: mol_type = other RNA
  modified_base 7: mod_base = m6a
  modified_base 1..4: mod_base = OTHER; note = 2' OMe linkage
  modified_base 9..15: mod_base = OTHER; note = 2' OMe linkage
aaaaggactgctttt

SEQ ID NO: 60 - DNA, 15 bp, synthetic construct
  misc_feature 1..15: note = Synthetic
  source 1..15: mol_type = other DNA
  modified_base 7: mod_base = m6a
aaaaggactgctttt

SEQ ID NO: 61 - DNA, 15 bp, synthetic construct
  source 1..15: mol_type = other DNA
  modified_base 7: mod_base = m6a
  misc_feature 1..6: note = DNA bases
  misc_feature 7: note = RNA base
  misc_feature 8..15: note = DNA bases
aaaaggactgctttt

SEQ ID NO: 62 - DNA, 15 bp, synthetic construct
  source 1..15: mol_type = other DNA
  modified_base 7: mod_base = m6a
  misc_feature 1..4: note = DNA bases
  misc_feature 5..9: note = RNA bases
  misc_feature 10..15: note = DNA bases
  misc_feature 9: note = Base is a U base and not a T
aaaaggactgctttt

SEQ ID NO: 63 - protein, 579 aa, Homo sapiens
MSASSLLEQRPKGQGNKVQNGSVHQKDGLNDDDFEPYLSPQARPNNAYTAMSDSYLPSYYSPSIGFSYSLGEAAWSTGGDTAMPYLTSYGQLSNGEPHFLPDAMFGQPGALGSTPFLGQHGFNFFPSGIDFSAWGNNSSQGQSTQSSGYSSNYAYAPSSLGGAMIDGQSAFANETLNKAPGMNTIDQGMAALKLGSTEVASNVPKVVGSAVGSGSITSNIVASNSLPPATIAPPKPASWADIASKPAKQQPKLKTKNGIAGSSLPPPPIKHNMDIGTWDNKGPVAKAPSQALVQNIGQPTQGSPQPVGQQANNSPPVAQASVGQQTQPLPPPPPQPAQLSVQQQAAQPTRWVAPRNRGSGFGHNGVDGNGVGQSQAGSGSTPSEPHPVLEKLRSINNYNPKDFDWNLKHGRVFIIKSYSEDDIHRSIKYNIWCSTEHGNKRLDAAYRSMNGKGPVYLLFSVNGSGHFCGVAEMKSAVDYNTCAGVWSQDKWKGRFDVRWIFVKDVPNSQLRHIRLENNENKPVTNSRDTQEVPLEKAKQVLKIIASYKHTTSIFDDFSHYEKRQEEEESVKKERQGRGK

SEQ ID NO: 64 - RNA, 20 nt, synthetic construct
  source 1..20: mol_type = other RNA
cccttctgtctccacctccc

SEQ ID NO: 65 - RNA, 15 nt, synthetic construct
  source 1..15: mol_type = other RNA
  modified_base 13: mod_base = m6a
ctgggaaactggact

SEQ ID NO: 66 - RNA, 18 nt, synthetic construct
  source 1..18: mol_type = other RNA
  modified_base 13: mod_base = m6a
ctgggaaactggactgag

SEQ ID NO: 67 - DNA, 18 bp, synthetic construct
  misc_feature 1..10: note = RNA
  misc_feature 11..15: note = DNA
  misc_feature 16..18: note = RNA
  source 1..18: mol_type = other DNA
  modified_base 1..18: mod_base = OTHER; note = Phosphorothioate linkage
  modified_base 13: mod_base = m6a
  modified_base 1..10: mod_base = OTHER; note = 2'O methyl modification
  modified_base 16..18: mod_base = OTHER; note = 2'O methyl modification
ctgggaaactggactgag

SEQ ID NO: 68 - DNA, 18 bp, synthetic construct
  misc_feature 1..10: note = RNA
  misc_feature 11..15: note = DNA
  misc_feature 16..18: note = RNA
  source 1..18: mol_type = other DNA
  modified_base 1..18: mod_base = OTHER; note = phosphorothioate linkage
  modified_base 13: mod_base = m6a
  modified_base 1..3: mod_base = OTHER; note = 2'O methyl modification
  modified_base 8..10: mod_base = OTHER; note = 2'O methyl modification
  modified_base 16..18: mod_base = OTHER; note = 2'O methyl modification
ctgggaaactggactgag

SEQ ID NO: 69 - RNA, 18 nt, synthetic construct
  source 1..18: mol_type = other RNA
  modified_base 1..18: mod_base = OTHER; note = phosphorothioate linkage
  modified_base 13: mod_base = m6a
  modified_base 1..3: mod_base = OTHER; note = 2'O methyl modification
  modified_base 8..12: mod_base = OTHER; note = 2'O methyl modification
  modified_base 14..18: mod_base = OTHER; note = 2'O methyl modification
ctgggaaactggactgag

SEQ ID NO: 70 - DNA, 18 bp, synthetic construct
  misc_feature 1..10: note = RNA
  misc_feature 11..15: note = DNA
  misc_feature 16..18: note = RNA
  source 1..18: mol_type = other DNA
  modified_base 1..18: mod_base = OTHER; note = phosphorothioate linkage
  modified_base 13: mod_base = m6a
  modified_base 1..10: mod_base = OTHER; note = 2-methoxyethyl modification
  modified_base 16..18: mod_base = OTHER; note = 2-methoxyethyl modification
ctgggaaactggactgag

SEQ ID NO: 71 - RNA, 18 nt, synthetic construct
  source 1..18: mol_type = other RNA
  modified_base 1..18: mod_base = OTHER; note = phosphorothioate linkage
  modified_base 13: mod_base = m6a
  modified_base 1..12: mod_base = OTHER; note = 2-methoxyethyl modification
  modified_base 14..18: mod_base = OTHER; note = 2-methoxyethyl modification
ctgggaaactggactgag

SEQ ID NO: 72 - DNA, 18 bp, synthetic construct
  misc_feature 1..10: note = RNA
  misc_feature 11..15: note = DNA
  misc_feature 16..18: note = RNA
  source 1..18: mol_type = other DNA
  modified_base 1..18: mod_base = OTHER; note = phosphorothioate linkage
  modified_base 13: mod_base = m6a
  modified_base 1..3: mod_base = OTHER; note = 2-methoxyethyl modification
  modified_base 8..10: mod_base = OTHER; note = 2-methoxyethyl modification
  modified_base 16..18: mod_base = OTHER; note = 2-methoxyethyl modification
ctgggaaactggactgag

SEQ ID NO: 73 - DNA, 25 bp, synthetic construct
  source 1..25: mol_type = other DNA
ccggaattcatgctcagacctatct

SEQ ID NO: 74 - DNA, 19 bp, synthetic construct
  source 1..19: mol_type = other DNA
cccatttcaggagacctgg